(19) United States
(12) Patent Application Publication
(10) Pub. No.: US 2012/0316184 A1
(43) Pub. Date: Dec. 13, 2012

(54) NOVEL MICROBICIDES

(75) Inventors: Martin Pouliot, Stein (CH); David Guillaume Claude Francois Lefranc, Stein (CH); Laura Quaranta, Stein (CH); Clemens Lamberth, Stein (CH); Nityakalyani Srinivas, Corlim (IN)

(73) Assignee: SYNGENTA CROP PROTECTION LLC, Greensboro, NC (US)

(21) Appl. No.: 13/581,272

(22) PCT Filed: Feb. 18, 2011
(86) PCT No.: PCT/EP2011/052418

§ 371 Date: Aug. 24, 2012

(30) Foreign Application Priority Data
Feb. 24, 2010 (IN) 402/DEL/10

(51) Int. Cl. A01N 43/90; A01P 1/00; C07D 471/04; C07D 513/04; C07D 498/04; C07D 495/04

(52) U.S. Cl. 514/260.1; 514/264.1; 514/267; 544/250; 544/255; 544/278; 544/279

(57) ABSTRACT

Compounds of formula (I) wherein G₁ represents together with the two ring atoms of the pyrimidine ring to which it is attached, a 5- to 6-membered aromatic heterocyclic ring system which contains one or two heteroatoms selected from the group consisting of nitrogen, oxygen and sulfur, and the other substituents are as defined in claim 1, are suitable for use as micro-biocides.

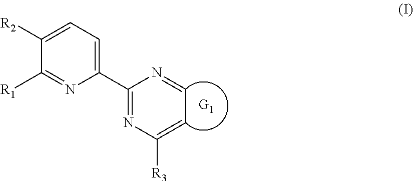

The present invention relates to novel microbiocidally active, in particular fungicidally active, 2-(pyridin-2-yl)-pyrimidines. It further relates to compositions which comprise these compounds and to their use in agriculture or horticulture for controlling or preventing infestation of plants by phytopathogenic microorganisms, preferably fungi.

Fungicidally active 2-(pyridin-2-yl)-pyrimidines are described in WO 2007/116079. The disclosed compounds are characterised by a condensed aliphatic carbocycle or heterocycle.

It has been found that novel 2-(pyridin-2-yl)-pyrimidines with a condensed aromatic heterocycle have microbiocidal activity.

The present invention accordingly relates to compounds of formula I

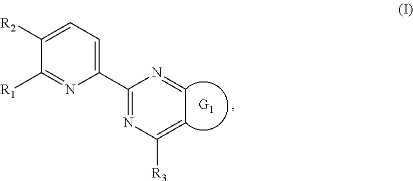

wherein
G₁ represents together with the two ring atoms of the pyrimidine ring to which it is attached, a 5- to 6-membered aromatic heterocyclic ring system which contains one or two heteroatoms selected from the group consisting of nitrogen, oxygen and sulfur; and wherein said 5- to 6-membered aromatic heterocyclic ring system can be mono- or disubstituted by substituents selected from the group consisting of C₁-C₆alkyl, C₁-C₆alkoxy, C₁-C₆alkylthio and halogen or two adjacent substituents together represent a polymethylene group of the formula —(CH₂)ₘ— in which m is 3 or 4;
R₁ is C₁-C₆alkyl, C₁-C₆haloalkyl, or a group —X—R₄, wherein X is a bond, oxygen, sulfur, C₁-C₄alkylene, C₂-C₄alkenylene or C₂-C₄alkynylene;
R₂ is hydrogen, C₁-C₆alkyl, C₁-C₆haloalkyl, C₁-C₆alkoxy, C₁-C₆haloalkoxy or C₃-C₈cycloalkyl;
R₃ is hydrogen, hydroxy, halogen, C₁-C₆alkyl, C₁-C₆alkoxy, phenyl, benzyl; or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy; R₄ is phenyl which can be mono-, di- or trisubstituted by substituents selected from the group consisting of halogen, nitro, C₁-C₄alkyl, C₁-C₆haloalkyl, C₁-C₄alkoxy and C₁-C₆haloalkoxy;
or
R₄ is additionally C₂-C₆alkynyl if X is a bond;
and agronomically acceptable salts or isomers or structural isomers or stereoisomers ordiastereoisomers or enantiomers or tautomers or atropoisomers and N-oxides of those compounds,
with the proviso that the compounds 4-chloro-5-methyl-2-(6-methyl-2-pyridinyl)-Thieno[2,3-d]pyrimidine and 4-chloro-2-(6-methyl-2-pyridinyl)-Thieno[3,2-d]pyrimidine and 5-methyl-2-(6-methyl-2-pyridinyl)-Thieno[2,3-d]pyrimidin-4(1H)-one and 2-(6-methyl-2-pyridinyl)-Thieno[3,2-d]pyrimidin-4(1H)-one and 4-chloro-2-(6-methyl-2-pyridinyl)-7H-Pyrrolo[2,3-d]pyrimidine and 4-iodo-2-(6-methyl-2-pyridinyl)-7H-Pyrrolo[2,3-d]pyrimidine and 2-(6-methyl-2-pyridinyl)-4(3H)-Pteridinone and 4-chloro-2-(6-methyl-2-pyridinyl)-Thieno[3,2-d]pyrimidine and 4-chloro-5-methyl-2-(6-methyl-2-pyridinyl)-Thieno[2,3-d]pyrimidine and 2-(6-methyl-2-pyridinyl)-Thieno[3,2-d]pyrimidin-4(3H)-one and 5-methyl-2-(6-methyl-2-pyridinyl)-Thieno[2,3-d]pyrimidin-4(1H)-one are excluded from the scope of protection.

The invention covers all agronomically acceptable salts/isomers/structural isomers/stereoisomers/diastereoisomers/enantiomers/tautomers and N-oxides of those compounds.

The alkyl groups occurring in the definitions of the substituents can be straight-chain or branched and are, for example, methyl, ethyl, n-propyl, n-butyl, n-pentyl, n-hexyl, iso-propyl, n-butyl, sec-butyl, iso-butyl or tert-butyl. Alkoxy, alkenyl and alkynyl radicals are derived from the alkyl radicals mentioned. The alkenyl and alkynyl groups can be mono- or di-unsaturated. The cycloalkyl groups are, for example, cyclopropyl, cyclobutyl, cyclopentyl or cyclohexyl. Halogen is generally fluorine, chlorine, bromine or iodine, preferably fluorine, bromine or chlorine. This also applies, correspondingly, to halogen in combination with other meanings, such as halogenalkyl or halogenalkoxy. Haloalkyl groups preferably have a chain length of from 1 to 4 carbon atoms. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably trichloromethyl, difluorochloromethyl, difluoromethyl, trifluoromethyl and dichlorofluoromethyl. Alkoxy is, for example, methoxy, ethoxy, propoxy, i-propoxy, n-butoxy, isobutoxy, sec-butoxy and tert-butoxy; preferably methoxy and ethoxy. Halogenalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy and 2,2,2-trichloroethoxy; preferably difluoromethoxy, 2-chloroethoxy and trifluoromethoxy.

Examples for G₁ which represents together with the two ring atoms of the pyrimidine ring to which it is attached, a 5- to 6-membered aromatic heterocyclic ring system which contains one or two heteroatoms selected from the group consisting of nitrogen, oxygen and sulfur are furane, pyrrole, thiophene, imidazole, pyrazole, isoxazole, pyridine, pyrazine, pyrimidine, pyridazine, thiazole, isothiazole or oxazole.

Preferred ring systems for the compounds of formula I formed by the pyrimidine ring together with the substituent G₁ are selected from the group consisting of

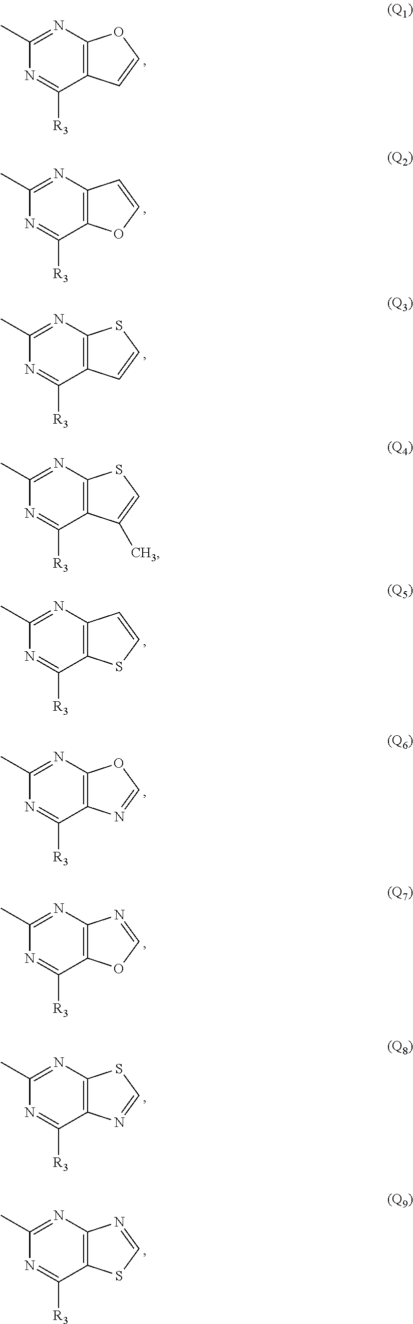

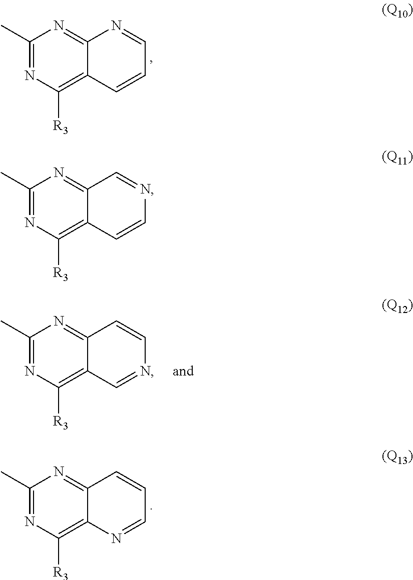

wherein R₃ has the meaning as defined for formula I above. In particular R₃ has the meaning of hydrogen, C₁-C₄alkyl, hydroxy, halogen or C₁-C₄alkoxy. More preferred rings systems are Q₃, Q₄. Q₁₀, Q₁₁, Q₁₂ and Q₁₃. Especially preferred rings systems are Q₃, Q₄. Q₁₃ and Q₁₀. Further especially preferred rings systems are Q₁₁ and Q₁₂.

By preference if R₁ is C₁-C₆alkyl, C₁-C₆haloalkyl, then R₃ is not hydrogen. By preference if R₁ is C₁-C₆alkyl, C₁-C₆haloalkyl, then R₃ is phenyl, benzyl; or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy. By preference if R₁ is a group —X—R₄, wherein X is a bond, oxygen, sulfur, C₁-C₄alkylene, C₂-C₄alkenylene or C₂-C₄alkynylene wherein R₄ is phenyl which can be mono-, di- or trisubstituted by substituents selected from the group consisting of halogen, nitro, C₁-C₄alkyl, C₁-C₆haloalkyl, C₁-C₄alkoxy and C₁-C₆haloalkoxy; or R₄ is additionally C₂-C₆alkynyl if X is a bond;; then R₃ is hydrogen.

Preferably R₁ is methyl or a group —X—R₄, wherein X is a bond, oxygen, sulfur, C₁-C₄alkylene, C₂-C₄alkenylene or C₂-C₄alkynylene; and R₄ is phenyl which can be mono-, di- or trisubstituted by substituents selected from the group consisting of halogen, nitro, C₁-C₄alkyl, C₁-C₆haloalkyl, C₁-C₄alkoxy and C₁-C₆haloalkoxy; or

R₄ is additionally C₂-C₆alkynyl if X is a bond;

By preference if R₁ is methyl then R₃ is not hydrogen.

By preference if R₁ is methyl then R₃ is phenyl, benzyl; or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy.

By preference if R₁ is a group —X—R₄, wherein X is a bond, oxygen, sulfur, C₁-C₄alkylene, C₂-C₄alkenylene or C₂-C₄alkynylene wherein R₄ is phenyl which can be mono-, di- or trisubstituted by substituents selected from the group consisting of halogen, nitro, C₁-C₄alkyl, C₁-C₆haloalkyl, C₁-C₄alkoxy and C₁-C₆haloalkoxy; or R₄ is additionally C₂-C₆alkynyl if X is a bond;; then R₃ is hydrogen.

In a preferred embodiment of the invention,

R₁ is a group —X—R₄, wherein X is a bond or C₁-C₄alkylene.

In another preferred embodiment of the invention, R₁ is methyl

In another preferred embodiment of the invention R₄ is phenyl which can be mono- or di- or trisubstituted by substituents selected from the group consisting of halogen, nitro, C₁-C₄alkyl, C₁-C₆haloalkyl, C₁-C₄alkoxy and C₁-C₆haloalkoxy.

In another preferred embodiment of the invention, R₁ is methyl R₃ is phenyl, benzyl; or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy.

In a preferred group of compounds of formula I, R₁ is a group —X—R₄, wherein X is a bond or C₁-C₄alkylene; and R₄ is phenyl which can be mono- or di- or trisubstituted by substituents selected from the group consisting of halogen, nitro, C₁-C₄alkyl, C₁-C₆haloalkyl, C₁-C₄alkoxy and C₁-C₆haloalkoxy.

In a further preferred group of compounds of formula I, R₁ is benzyl or phenyl which can be substituted by halogen or C₁-C₄alkoxy; R₂ is hydrogen or C₁-C₄alkyl; R₃ is hydrogen, C₁-C₄alkyl, hydroxy, halogen or C₁-C₄alkoxy.

In a further preferred group of compounds of formula I, R₁ is phenyl which can be substituted by halogen or C₁-C₄alkoxy; R₂ is hydrogen or C₁-C₄alkyl; R₃ is hydrogen, C₁-C₄alkyl, hydroxy, halogen or C₁-C₄alkoxy.

In a further preferred group of compounds of formula I, R₁ is methyl; R₂ is hydrogen or C₁-C₄alkyl; R₃ is hydrogen, hydroxy, halogen, C₁-C₄alkyl, C₁-C₄alkoxy or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;

In a further preferred group of compounds of formula I, R₁ is methyl; R₂ is hydrogen or C₁-C₄alkyl; R₃ is C₁-C₄alkyl, C₁-C₄alkoxy or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;

In a further preferred group of compounds of formula I, R₁ is methyl; R₂ is hydrogen or C₁-C₄alkyl; R₃ is phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;

In a further preferred group of compounds of formula I, R₁ is methyl; R₂ is hydrogen or C₁-C₄alkyl; R₃ is phenyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;

Further preferred embodiments of the present invention are the embodiments E1 to E13, which are defined as compounds of formula I which are represented by one formula selected from the group consisting of the formulae T1 to T13 as described below, wherein in formulae T1 to T13:

R₁ is phenoxy, phenylthio, benzyl, which itself can be mono- or disubstituted by substituents selected from halogen, C₁-C₄alkyl and C₁-C₄alkoxy; or is C₆H₅CH═CH, C₆H₅C≡C, phenyl, or phenyl which is substituted by substituents selected from halogen, C₁-C₄alkyl and C₁-C₄alkoxy;

R₂ is hydrogen or C₁-C₄alkyl, and
R₃ is hydrogen, hydroxy, halogen, C₁-C₄alkyl or C₁-C₄alkoxy.

For example, embodiment E1 is represented by the compounds of formula T1

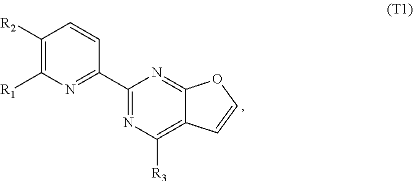

wherein
R₁ is phenoxy, phenylthio, benzyl, which itself can be mono- or disubstituted by substituents selected from halogen, C₁-C₄alkyl and C₁-C₄alkoxy; or is C₆H₅CH═CH, C₆H₅C≡C, phenyl, or phenyl which is substituted by substituents selected from halogen, C₁-C₄alkyl and C₁-C₄alkoxy;
R₂ is hydrogen or C₁-C₄alkyl; and
R₃ is hydrogen, hydroxy, halogen, C₁-C₄alkyl or C₁-C₄alkoxy.

Embodiments E2 to E13 are defined accordingly. Preferred embodiments are embodiment E3, E4 and E10, in particular E3. Within said embodiments E1 to E13, preferably E3, E4, E10, E11 and E13, especially preferred embodiments are E3, E4. E13 and E10 and further especially embodiments are E11 and E12, in particular E3, the following meanings of the substituents are preferred:

In a preferred embodiment E1 to E13, preferably E3, E4, E10, E11 and E13, wherein especially preferred embodiments are E3, E4. E13 and E10 and further especially preferred rings systems are E11 and E12, in particular E3, R₁ is a group —X—R₄, wherein X is a bond or C₁-C₄alkylene; and R₄ is phenyl which can be mono- or di- or trisubstituted by substituents selected from the group consisting of halogen, nitro, C₁-C₄alkyl, C₁-C₆haloalkyl, C₁-C₄alkoxy and C₁-C₆haloalkoxy.

In a preferred embodiment E1 to E13, preferably E3, E4, E10, E11 and E13, wherein especially preferred embodiments are E3, E4. E13 and E10 and further especially preferred rings systems are E11 and E12, in particular E3, R₁ is benzyl or phenyl which can be substituted by halogen, C₁-C₄alkyl or C₁-C₄alkoxy; R₂ is hydrogen or C₁-C₄alkyl; R₃ is hydrogen, C₁-C₄alkyl, hydroxy, halogen or C₁-C₄alkoxy.

In a preferred embodiment E1 to E13, preferably E3, E4, E10, E11 and E13, wherein especially preferred embodiments are E3, E4. E13 and E10 and further especially preferred rings systems are E11 and E12, in particular E3, R₁ is phenyl which can be substituted by halogen, C₁-C₄alkyl or C₁-C₄alkoxy;

R₂ is hydrogen or C₁-C₄alkyl; R₃ is hydrogen, C₁-C₄alkyl, hydroxy, halogen or C₁-C₄alkoxy.

In a preferred embodiment E1 to E13, preferably E3, E4, E10, E11 and E13, wherein especially preferred embodiments are E3, E4. E13 and E10 and further especially preferred rings systems are E11 and E12, in particular E3, R₁ is methyl; R₂ is hydrogen or C₁-C₄alkyl; R₃ is hydrogen, C₁-C₄alkyl, hydroxy, halogen, C₁-C₄alkoxy or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;

In a preferred embodiment E1 to E13, preferably E3, E4, E10, E11 and E13, wherein especially preferred embodiments are E3, E4. E13 and E10 and further especially preferred rings systems are E11 and E12, in particular E3, R₁ is methyl; R₂ is hydrogen or C₁-C₄alkyl; R₃ is C₁-C₄alkyl, C₁-C₄alkoxy or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;

In a preferred embodiment E1 to E13, preferably E3, E4, E10, E11 and E13, wherein especially preferred embodiments are E3, E4. E13 and E10 and further especially preferred rings systems are E11 and E12, in particular E3, R₁ is methyl; R₂ is hydrogen or C₁-C₄alkyl; R₃ is phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;

In a preferred embodiment E1 to E13, preferably E3, E4, E10, E11 and E13, wherein especially preferred embodiments are E3, E4. E13 and E10 and further especially preferred rings systems are E11 and E12, in particular E3, R₁ is methyl; R₂ is hydrogen or C₁-C₄alkyl; R₃ is phenyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;

Compounds of formula I may be prepared as shown in the following schemes.

The compounds of formula I.1, wherein R₁ and R₂ are as defined under formula I, can be obtained by transformation of a compound of formula II, wherein R₁ and R₂ are as defined under formula I, with an oxidation agent, such as 2,3-dichloro-5,6-dicycano-p-benzoquinone, oxygen, manganese(IV) oxide or ammonium cerium(IV) nitrate.

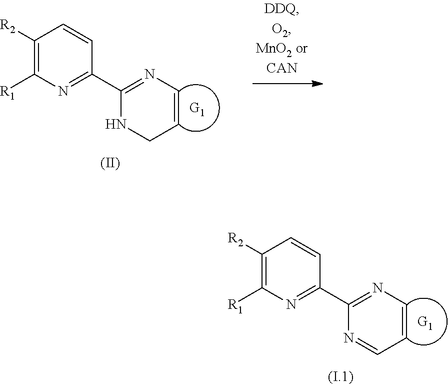

The compounds of formula II, wherein R₁ and R₂ are as defined under formula I, can be obtained by transformation of a compound of formula I.2, wherein R₁ and R₂ are as defined under formula I and Hal is halogen, preferably chlorine or bromine, with a reduction agent such as hydrogen and a catalyst such as palladium on charcoal or raney-nickel.

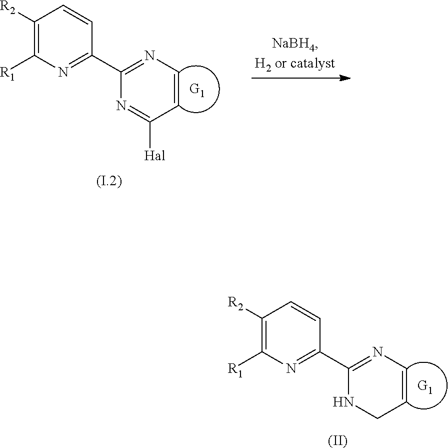

The compounds of formula I.2, wherein R₁ and R₂ are as defined under formula I and Hal is halogen, preferably chlorine or bromine, can be obtained by transformation of a compound of formula III, wherein R₁ and R₂ are as defined under formula I with a phosphorus oxyhalide, e.g. phosphorus oxychloride or phosphorus oxybromide, or a thionyl halide, e.g. thionyl chloride or thionyl bromide.

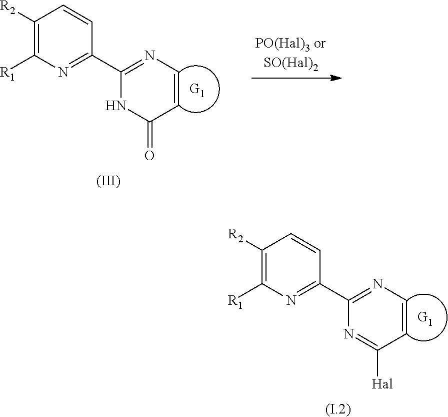

The compounds of formula III, wherein R₁ and R₂ are as defined under formula I, can be obtained by transformation of a compound of formula IV, wherein R₁ and R₂ are as defined under formula I, with a compound of formula V and a base, such as sodium hydride, sodium methylate, sodium ethylate or potassium methylate.

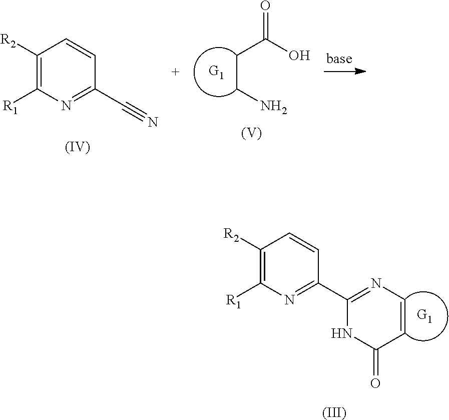

The beta-amino acids of formula V are known compounds or may be obtained readily from known compounds using processes that are routine in the art and with which the skilled man will be familiar.

The compounds of formula IV, wherein R₁ and R₂ are as defined under formula I can be obtained by transformation of a compound of formula VI, wherein R₁ and R₂ are as defined under formula I with a cyanide, such as sodium cyanide, potassium cyanide or trimethylsilylcyanide and a base, such as triethylamine, ethyldiisopropylamine or pyridine.

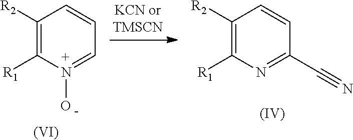

The compounds of formula VI, wherein R₁ and R₂ are as defined under formula I, can be obtained by transformation of a compound of formula VII, wherein R₁ and R₂ are as defined under formula I, with an oxidatizing agent, such as meta-chloroperbenzoic acid, hydrogen peroxide or oxone.

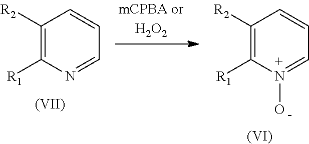

The mono- and disubstituted pyridines of formula VII are known compounds or may be obtained readily from known compounds using processes that are routine in the art and with which the skilled man will be familiar.

Alternatively, the compounds of formula III, wherein R₁ and R₂ are as defined under formula I, can be obtained by transformation of a compound of formula VIII, wherein R₁ and R₂ are as defined under formula I with a beta-amino acid amide of formula IX and a base, such as sodium hydride, sodium methylate, sodium ethylate or potassium methylate.

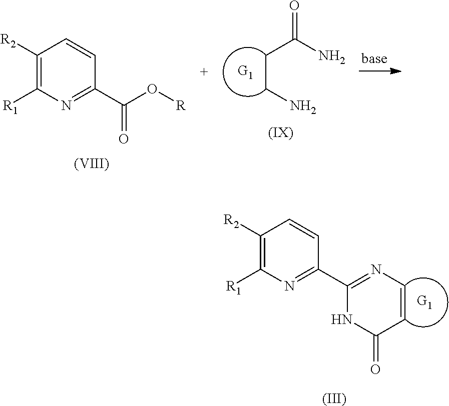

Alternatively, the compounds of formula I.1, wherein R₁ and R₂ are as defined under formula I, can be obtained by transformation of a compound of formula X, wherein R₁ and R₂ are as defined under formula I with an oxidation agent, such as 2,3-dichloro-5,6-dicycano-p-benzoquinone, oxygen, manganese(IV) oxide or ammonium cerium(IV) nitrate.

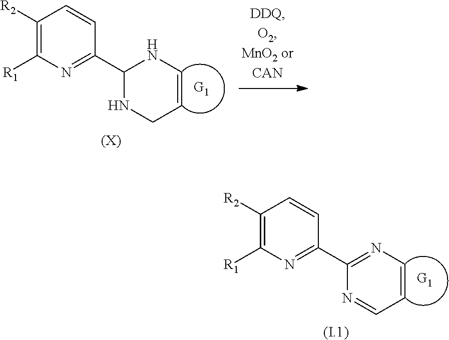

The compounds of formula X, wherein R₁ and R₂ are as defined under formula I, can be obtained by transformation of a compound of formula XI, wherein R₁ and R₂ are as defined under formula I, with a diamine of formula XII and thionyl chloride and a base, such as triethylamine, ethyldiisopropylamine or pyridine.

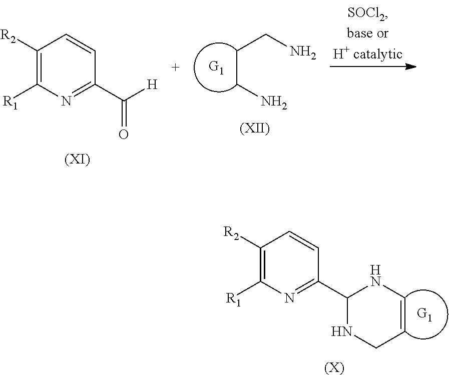

The diamines of formula XII are known compounds or may be obtained readily from known compounds using processes that are routine in the art and with which the skilled man will be familiar.

The compounds of formula XI, wherein R₁ and R₂ are as defined under formula I, can be obtained by transformation of a compound of formula XIII, wherein R₁ and R₂ are as defined under formula I, with N,N′-dicyclohexylcarbodiimide, dimethylsulfoxide and an acid, such as phosphoric acid, hydrochloric acid or sulfuric acid, or with manganese dioxide or 2,3-dichloro-5,6-dicycano-p-benzoquinone.

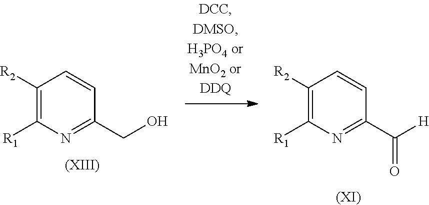

The compounds of formula XIII, wherein R₁ and R₂ are as defined under formula I, can be obtained by transformation of a compound of formula VIII, wherein R₁ and R₂ are as defined under formula I and R₅ is hydrogen or C₁-C₆alkyl, with an reducing agent, such as sodium borohydride, lithium aluminium hydride, lithium borohydride or diisobutylaluminum hydride.

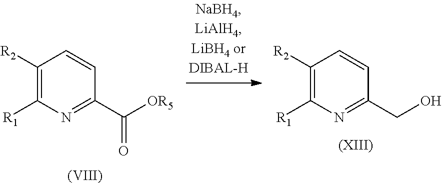

Alternatively the compounds of formula I.1, wherein R₁ and R₂ are as defined under formula I, can be obtained by transformation of a compound of formula XIV, wherein R₁ and R₂ are as defined under formula I, or a salt of it, with an aldehyde of formula XV, wherein R₆ is a halogen, such as fluoro, or an amino group, and a base, such as sodium carbonate, sodium bicarbonate or potassium carbonate.

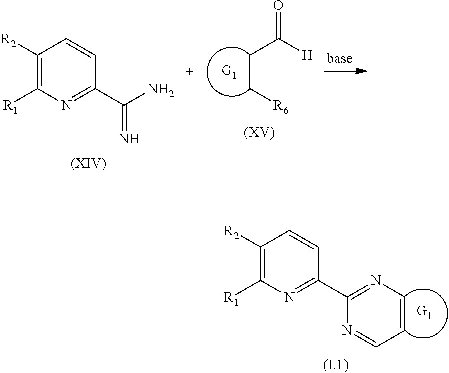

The aldehydes of formula XV are known compounds or may be obtained readily from known compounds using processes that are routine in the art and with which the skilled man will be familiar.

The compounds of formula XIV, wherein R₁ and R₂ are as defined under formula I can be obtained by transformation of a compound of formula IV, wherein R₁ and R₂ are as defined under formula I with a base and an ammonium salt.

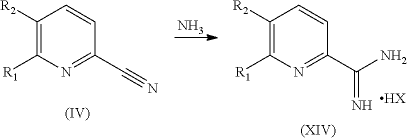

The compounds of formula I.3, wherein R₁ and R₂ are as defined under formula I and R₇ is C₁₋₈ alkyl can be obtained by alkylation of a compound of formula I.2, wherein R₁ and R₂ are as defined under formula I and Hal is halogen, preferably chlorine or bromine, with an alcohol R₇—OH, wherein R₇ is C₁₋₆ alkyl, and a base, such as sodium hydride, potassium hydride, sodium carbonate, potassium carbonate, sodium hydroxide or potassium hydroxide.

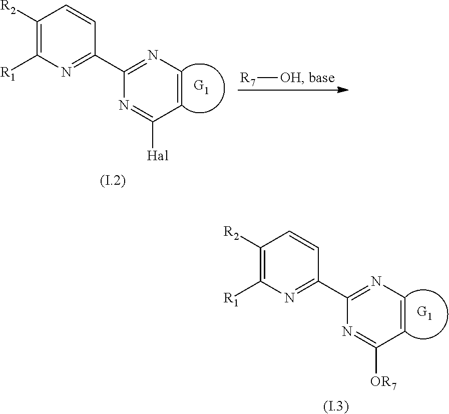

Alternatively the compounds of formula I.3, wherein R₁ and R₂ are as defined under formula I and R₇ is C₁₋₆ alkyl, can be obtained by alkylation of a compound of formula III, wherein R₁ and R₂ are as defined under formula I with a compound R₇-Hal, wherein R₇ is C₁₋₆ alkyl and Hal is halogen, preferably chlorine or bromine, and a base, such as sodium hydride, potassium hydride, sodium carbonate, potassium carbonate, sodium hydroxide or potassium hydroxide.

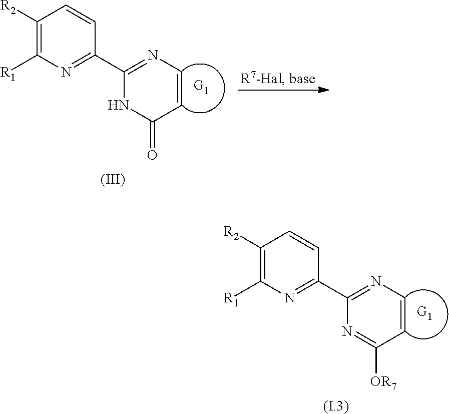

The compounds of formula I.4, wherein R₁ and R₂ are as defined under formula I and R₇ is C₁₋₈ alkyl, can be obtained by alkylation of a compound of formula I.2, wherein R₁ and R₂ are as defined under formula I and Hal is halogen, preferably chlorine or bromine, with an organometallic species, such as methylmagnesium chloride, methylmagnesium bromide or trimethylaluminum.

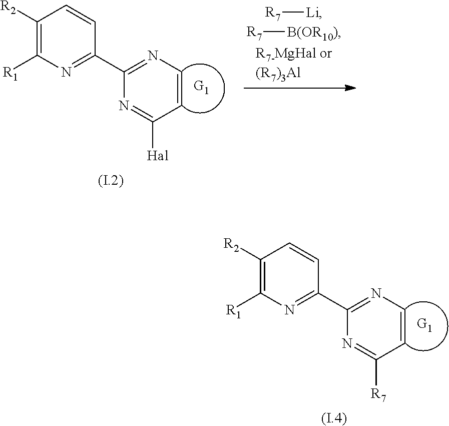

Alternatively, the compounds of formula I, wherein R₁, R₂ and R₃ are as defined under formula I, can be obtained by transformation of a compound of formula XVI, wherein R₁ and R₂ are as defined under formula I and R₉ is In, MgCl, MgBr, SnBu₃, ZnCl, ZnBr or B(OR₁₀)₂, wherein each R₁₀ independently is hydrogen or C₁-C₆alkyl or together forms a four to six-membered saturated ring, with a compound of formula XVII, wherein R₃ is as defined under formula I and R₉ is a halogen, preferably chloro, bromo or iodo or a sulfonic ester such as a mesylate or tosylate and a catalyst, such as tetrakistriphenylphosphinepalladium, palladium dichloride, [1,1-bis(diphenylphosphino)ferrocene]dichloropalladium(II), palladium acetate or bis(diphenylphosphine)palladium(II) chloride.

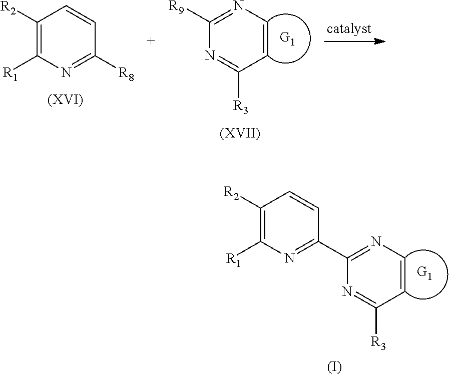

The metallo-substituted pyridines of formula XVI and the 2-halopyrimidines of formula XVII are known compounds or may be obtained readily from known compounds using processes that are routine in the art and with which the skilled man will be familiar.

Alternatively, the compounds of formula I, wherein R₁, R₂ and R₃ are as defined under formula I, can be obtained by transformation of a compound of formula XVIII, wherein R₁ and R₂ are as defined under formula I and R₉ is a halogen, preferably chloro, bromo or iodo or a sulfonic ester such as a mesylate or tosylate, with a compound of formula XIV, wherein R₃ is as defined under formula I and R₈ is In, MgCl, MgBr, SnBu₃, ZnCl, ZnBr or B(OR₁₀)₂, wherein each R₁₀ independently is hydrogen or C₁-C₆alkyl or together forms a four to six-membered saturated ring, and a catalyst, such as tetrakistriphenylphosphinepalladium, palladium dichloride, [1,1-bis(diphenylphosphino)ferrocene]dichloropalladium(II), palladium acetate or bis(diphenylphosphine)palladium(II) chloride.

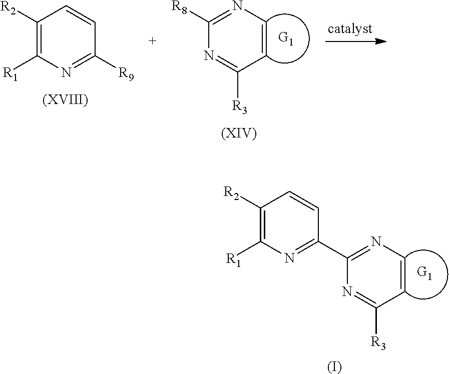

The di- and tri-substituted pyridines of formula XVIII and the 2-metallo-substituted pyrimidines of formula XIV are known compounds or may be obtained readily from known compounds using processes that are routine in the art and with which the skilled man will be familiar.

The reactions to give compounds of formula I are advantageously carried out in aprotic inert organic solvents. Such solvents are hydrocarbons such as benzene, toluene, xylene or cyclohexane, chlorinated hydrocarbons such as dichloromethane, trichloromethane, tetrachloromethane or chlorobenzene, ethers such as diethyl ether, ethylene glycol dimethyl ether, diethylene glycol dimethyl ether, tetrahydrofuran or dioxane, nitriles such as acetonitrile or propionitrile, amides such as N,N-dimethylformamide, diethylformamide or N-methylpyrrolidinone. The reaction temperatures are advantageously between −20° C. and +120° C. In general, the reactions are slightly exothermic and, as a rule, they can be carried out at ambient temperature. To shorten the reaction time, or else to start the reaction, the mixture may be heated briefly to the boiling point of the reaction mixture. The reaction times can also be shortened by adding a few drops of base as reaction catalyst. Suitable bases are, in particular, tertiary amines such as trimethylamine, triethylamine, quinuclidine, 1,4-diazabicyclo[2.2.2]octane, 1,5-diazabicyclo[4.3.0]non-5-ene or 1,5-diazabicyclo-[5.4.0]undec-7-ene. However, inorganic bases such as hydrides, e.g. sodium hydride or calcium hydride, hydroxides, e.g. sodium hydroxide or potassium hydroxide, carbonates such as sodium carbonate and potassium carbonate, or hydrogen carbonates such as potassium hydrogen carbonate and sodium hydrogen carbonate may also be used as bases. The bases can be used as such or else with catalytic amounts of a phase-transfer catalyst, for example a crown ether, in particular 18-crown-6, or a tetraalkylammonium salt.

The compounds of formula I and, where appropriate, the tautomers thereof, can, if appropriate, also be obtained in the form of hydrates and/or include other solvents, for example those which may have been used for the crystallization of compounds which are present in solid form.

It has now been found that the compounds of formula I according to the invention have, for practical purposes, a very advantageous spectrum of activities for protecting useful plants against diseases that are caused by phytopathogenic microorganisams, such as fungi, bacteria or viruses.

The invention therefore also relates to a method of controlling or preventing infestation of useful plants by phytopathogenic microorganisms, wherein a compound of formula I is applied as active ingredient to the plants, to parts thereof or the locus thereof. The compounds of formula I according to the invention are distinguished by excellent activity at low rates of application, by being well tolerated by plants and by being environmentally safe. They have very useful curative, preventive and systemic properties and are used for protecting numerous useful plants. The compounds of formula I can be used to inhibit or destroy the diseases that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula I as dressing agents for the treatment of plant propagation material, in particular of seeds (fruit, tubers, grains) and plant cuttings (e.g. rice), for the protection against fungal infections as well as against phytopathogenic fungi occurring in the soil.

Furthermore, the compounds of formula I according to the invention may be used for controlling fungi in related areas, for example in the protection of technical materials, including wood and wood related technical products, in food storage or in hygiene management.

The compounds of formula I are, for example, effective against the phytopathogenic fungi of the following classes: Fungi imperfecti (e.g. Botrytis, Pyricularia, Helminthosporium, Fusarium, Septoria, Cercospora and Alternaria) and Basidiomycetes (e.g. Rhizoctonia, Hemileia, Puccinia). Additionally, they are also effective against the Ascomycetes classes (e.g. Venturia and Erysiphe, Podosphaera, Monilinia, Uncinula) and of the Oomycetes classes (e.g. Phytophthora, Pythium, Plasmopara). Furthermore, the novel compounds of formula I are effective against phytopathogenic bacteria and viruses (e.g. against Xanthomonas spp, Pseudomonas spp, Erwinia amylovora as well as against the tobacco mosaic virus). The compounds of formula I are also effective against Asian soybean rust (Phakopsora pachyrhizi).

Within the scope of the invention, useful plants to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as ornamentals.

The term “useful plants” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “useful plants” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The term “locus” of a useful plant as used herein is intended to embrace the place on which the useful plants are growing, where the plant propagation materials of the useful plants are sown or where the plant propagation materials of the useful plants will be placed into the soil. An example for such a locus is a field, on which crop plants are growing.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The compounds of formula I can be used in unmodified form or, preferably, together with carriers and adjuvants conventionally employed in the art of formulation.

Therefore the invention also relates to compositions for controlling and protecting against phytopathogenic microorganisms, comprising a compound of formula I and an inert carrier, and to a method of controlling or preventing infestation of useful plants by phytopathogenic microorganisms, wherein a composition, comprising a compound of formula I as active ingredient and an inert carrier, is applied to the plants, to parts thereof or the locus thereof.

To this end compounds of formula I and inert carriers are conveniently formulated in known manner to emulsifiable concentrates, coatable pastes, directly sprayable or dilutable solutions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants (auxiliaries) can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula I or compositions, comprising a compound of formula I as active ingredient and an inert carrier, can be applied to the locus of the plant or plant to be treated, simultaneously or in succession with further compounds. These further compounds can be e.g. fertilizers or micronutrient donors or other preparations which influence the growth of plants. They can also be selective herbicides as well as insecticides, fungicides, bactericides, nematicides, molluscicides or mixtures of several of these preparations, if desired together with further carriers, surfactants or application promoting adjuvants customarily employed in the art of formulation.

A preferred method of applying a compound of formula I, or a composition, comprising a compound of formula I as active ingredient and an inert carrier, is foliar application. The frequency of application and the rate of application will depend on the risk of infestation by the corresponding pathogen. However, the compounds of formula I can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation, i.e. a composition comprising the compound of formula I and, if desired, a solid or liquid adjuvant, is prepared in a known manner, typically by intimately mixing and/or grinding the compound with extenders, for example solvents, solid carriers and, optionally, surface-active compounds (surfactants).

The agrochemical formulations will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Whereas it is preferred to formulate commercial products as concentrates, the end user will normally use dilute formulations.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient rates of application are from 10 mg to 1 g of active substance per kg of seeds. The rate of application for the desired action can be determined by experiments. It depends for example on the type of action, the developmental stage of the useful plant, and on the application (location, timing, application method) and can, owing to these parameters, vary within wide limits.

The present invention relates additionally to mixtures comprising at least a compound of formula I and at least a further, other biocidally active ingredient and optionally further ingredients. The further, other biocidally active ingredient are known for example from “The Pesticide Manual” [The Pesticide Manual—A World Compendium; Thirteenth Edition (New edition (2 Nov. 2003)); Editor: C. D. S. Tomlin; The British Crop Protection Council, ISBN-10: 1901396134; ISBN-13: 978-1901396133] or its electronic version “e-Pesticide Manual V4.2” or from the website http://www.alanwood.net/pesticides/ or preferably one of the further pesticides listed below.

The following mixtures of the compounds of TX with a further active ingredient (B) are preferred (the abbreviation “TX” means “one compound selected from the group consisting of the compounds of formulae from the lines A.1.1 to A.1.205 described in Tables 1 to 18 of the present invention, thus the abbreviation “TX” means at least one compound selected from the compounds T.1.1 to T18.205:

### (B)

(B1) a strobilurin fungicide+TX,
(B2) an azole fungicide+TX,
(B3) a morpholine fungicide+TX,
(B4) an anilinopyrimidine fungicide+TX,
(B5) a fungicide selected from the group consisting of
Anilazine+TX, arsenates+TX, benalaxyl+TX, benalaxyl-M+TX, benodanil+TX, benomyl+TX, benthiavalicarb+TX, benthiavalicarb-isopropyl+TX, biphenyl+TX, bitertanol+TX, blasticidin-S+TX, bordeaux mixture+TX, boscalid+TX, bupirimate+TX, cadmium chloride+TX, captafol+TX, captan+TX, carbendazim+TX, carbon disulfide+TX, carboxin+TX, carpropamid+TX, cedar leaf oil+TX, chinomethionat+TX, chlorine+TX, chloroneb+TX, chlorothalonil+TX, chlozolinate+TX, cinnamaldehyde+TX, copper+TX, copper ammoniumcarbonate+TX, copper hydroxide+TX, copper octanoate+TX, copper oleate+TX, copper sulphate+TX, cyazofamid+TX, cycloheximide+TX, cymoxanil+TX, dichlofluanid+TX, dichlone+TX, dichloropropene+TX, diclocymet+TX, diclomezine+TX, dicloran+TX, diethofencarb+TX, diflumetorim+TX, dimethirimol+TX, dimethomorph+TX, dinocap+TX, dithianon+TX, dodine+TX, edifenphos+TX, ethaboxam+TX, ethirimol+TX, etridiazole+TX, famoxadone+TX, fenamidone+TX, fenaminosulf+TX, fenamiphos+TX, fenarimol+TX, fenfuram+TX, fenhexamid+TX, fenoxanil+TX, fenpiclonil+TX, fentin acetate+TX, fentin chloride+TX, fentin hydroxide+TX, ferbam+TX, ferimzone+TX, fluazinam+TX, fludioxonil+TX, flusulfamide+TX, flusulfamide+TX, flutolanil+TX, folpet+TX, formaldehyde+TX, fosetyl-aluminium+TX, fthalide+TX, fuberidazole+TX, furalaxyl+TX, furametpyr+TX, flyodin+TX, fuazatine+TX, hexachlorobenzene+TX, hymexazole+TX, iminoctadine+TX, iodocarb+TX, iprobenfos+TX, iprodione+TX, iprovalicarb+TX, isoprothiolane+TX, kasugamycin+TX, mancozeb+TX, maneb+TX, manganous dimethyldithiocarbamate+TX, mefenoxam+TX, mepronil+TX, mercuric chloride+TX, mercury+TX, metalaxyl+TX, methasulfocarb+TX, metiram+TX, metrafenone+TX, nabam+TX, neem oil (hydrophobic extract)+TX, nuarimol+TX, octhilinone+TX, ofurace+TX, oxadixyl+TX, oxine copper+TX, oxolinic acid+TX, oxycarboxin+TX, oxytetracycline+TX, paclobutrazole+TX, paraffin oil+TX, paraformaldehyde+TX, pencycuron+TX, pentachloronitrobenzene+TX, pentachlorophenol+TX, penthiopyrad+TX, perfurazoate+TX, phosphoric acid+TX, polyoxin+TX, polyoxin D zinc salt+TX, potassium bicarbonate+TX, probenazole+TX, procymidone+TX, propamocarb+TX, propineb+TX, proquinazid+TX, prothiocarb+TX, pyrazophos+TX, pyrifenox+TX, pyroquilon+TX, quinoxyfen+TX, quintozene+TX, silthiofam+TX, sodium bicarbonate+TX, sodium diacetate+TX, sodium propionate+TX, streptomycin+TX, sulphur+TX, TCMTB+TX, tecloftalam+TX, tecnazene+TX, thiabendazole+TX, thifluzamide+TX, thiophanate+TX, thiophanate-methyl+TX, thiram+TX, tolclofos-methyl+TX, tolyfluanid+TX, triazoxide+TX, trichoderma harzianum+TX, tricyclazole+TX, triforine+TX, triphenyltin hydroxide+TX, validamycin+TX, vinclozolin+TX, zineb+TX, ziram+TX, zoxamide+TX, 1+TX, 1-bis(4-chlorophenyl)-2-ethoxyethanol+TX, 2+TX, 4-dichlorophenyl benzenesulfonate+TX, 2-fluoro-N-methyl-N-1-naphthylacetamide+TX, 4-chlorophenyl phenyl sulfone+TX,
a compound of formula B-5.1+TX

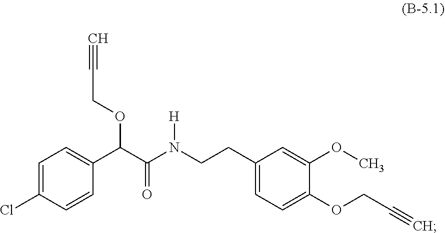

a compound of formula B-5.2+TX

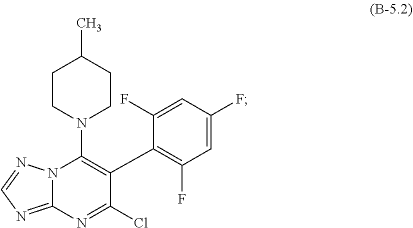

a compound of formula B-5.3+TX

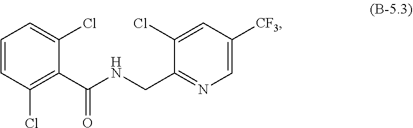

a compound of formula B-5.4+TX

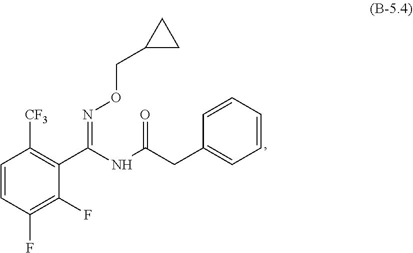

a compound of formula B-5.5+TX

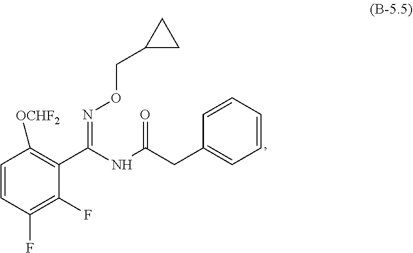

a compound of formula B-5.6+TX

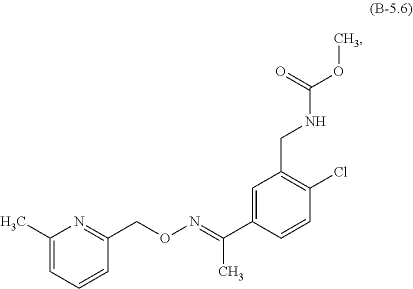

a compound of formula B-5.7+TX

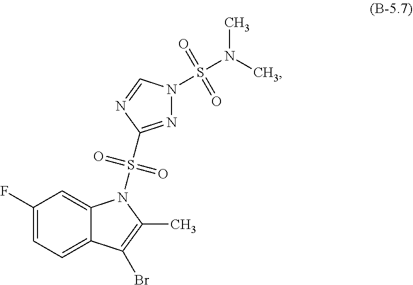

3-difluoromethyl-1-methyl-1H-pyrazole-4-carboxylic acid (2-bicyclopropyl-2-yl-phenyl)-amide (compound B-5.8)+TX, 3-difluoromethyl-1-methyl-1H-pyrazole-4-carboxylic acid (9-isopropyp-1,2,3,4-tetrahydro-1,4-methano-naphthalen-5-yl)-amide (compound B-5.9)+TX, 1,3-dimethyl-5-fluoro-1H-pyrazole-4-carboxylic acid [2-(1,3-dimethylbutyl)phenyl]-amide (compound B-5.10)+TX, 3-difluoromethyl-1-methyl-1H-pyrazole-4-carboxylic acid (3′,4′-dichloro-5-fluoro-1,1′-biphenyl-2-yl)-amide (compound B-5.11)+TX, N-{2-[3-chloro-5-(trifluoromethyl)pyridin-2-yl]ethyl}-2-(trifluoromethyl)benzamid (compound B-5.12)+TX, 3-difluoromethyl-1-methyl-1H-pyrazole-4-carboxylic acid N-[2-(1,1,2,2-tetrafluoroethoxy)phenyl]-amide (compound B-5.13)+TX, 3-difluoromethyl-1-methyl-1H-pyrazole-4-carboxylic acid N-[2-(1,1,2,3,3,3-hexafluoropropoxy)phenyl]-amide (compound B-5.14), 3-difluoromethyl-1-methyl-1H-pyrazole-4-carboxylic acid N-[2-(2-chloro-1+TX, 1,2-trifluoroethoxy)phenyl]-amide (compound B-5.15)+TX, 3-difluoromethyl-1-methyl-1H-pyrazole-4-carboxylic acid N-(4′-trifluoromethyl-biphen-2-yl)-amide (compound B-5.16)+TX, 3-difluoromethyl-1-methyl-1H-pyrazole-4-carboxylic acid N-(2′-trifluoromethyl-biphen-2-yl)-amide (compound B-5.17) and 3-difluoromethyl-1-methyl-1H-pyrazole-4-carboxylic acid N-(2′-trifluoromethyl-biphen-2-yl)-amide (compound B-5.18)+TX; (B6) a plant-bioregulator selected from the group consisting of
acibenzolar-5-methyl+TX, chlormequat chloride+TX, ethephon+TX, mepiquat chloride and trinexapc-ethyl;
(B7) an insecticide selected from the group consisting of
abamectin+TX, clothianidin+TX, emamectin benzoate+TX, imidacloprid+TX, tefluthrin+TX, thiamethoxam+TX,
and a compound of formula IV+TX

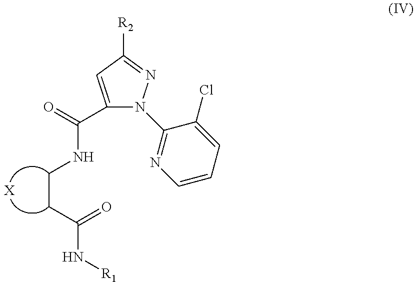

wherein X is a bivalent group selected from

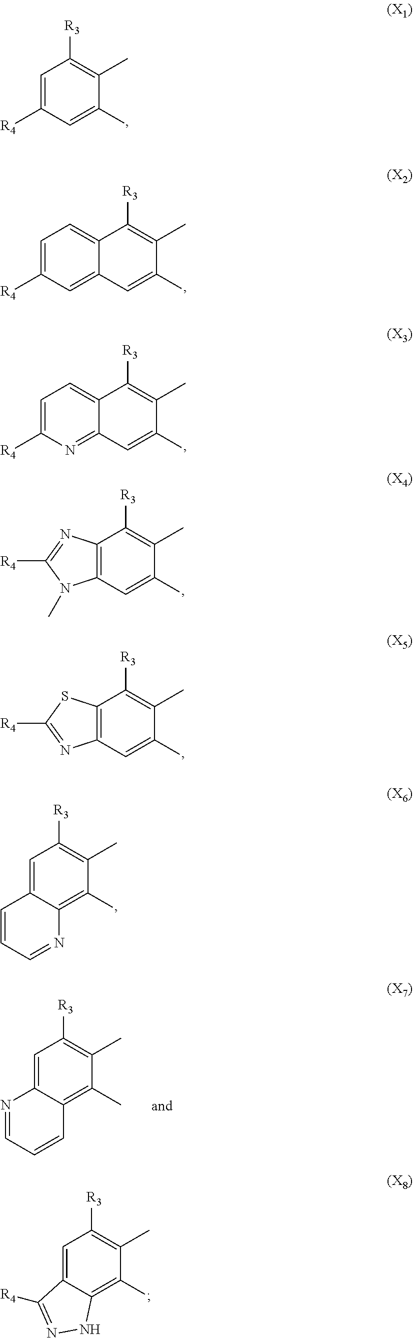

wherein
a) R₁ is cyclopropyl substituted by cyclopropyl at the 1-position, R₂ is bromine, R₃ is methyl, R₄ is CN and X is X₁;
b) R₁ is methyl substituted by cyclopropyl, R₂ is CF₃, R₃ is methyl, R₄ is Cl and X is X₁;
c) R₁ is cyclopropyl substituted by cyclopropyl at the 1-position, R₂ is CF₃, R₃ is methyl, R₄ is Cl and X is X₁;
d) R₁ is cyclopropyl substituted by cyclopropyl at the 1-position, R₂ is CF₃, R₃ is methyl, R₄ is CN and X is X₁;
e) R₁ is cyclopropyl substituted by cyclopropyl at the 1-position, R₂ is OCH₂CF₃, R₃ is methyl, R₄ is CN and X is X₁;
f) R₁ is isopropyl, R₂ is methoxy; R₃ is methyl, R₄ is hydrogen and X is X₈;
g) R₁ is isopropyl, R₂ is trifluoromethyl, R₃ is chlorine, R₄ is hydrogen and X is X₈;
h) R₁ is isopropyl, R₂ is trifluoromethyl, R₃ is methyl, R₄ is hydrogen and X is X₈;
i) R₁ is methyl, R₂ is bromine, R₃ is methyl, R₄ is CN and X is X₁;
j) R₁ is methyl, R₂ is bromine, R₃ is methyl, R₄ is Cl and X is X₁;
and (B8) glyphosate+TX, a compound of formula V+TX

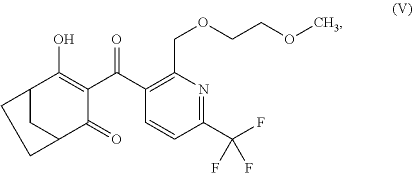

fomesafen+TX, and (B9) Isopyrazam+TX, Sedaxane+TX,
a compound of formula (VI)+TX

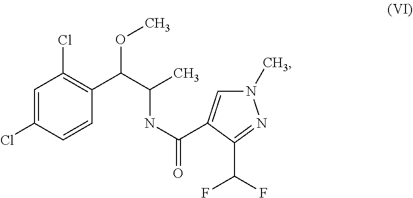

a compound of formula (VII)+TX

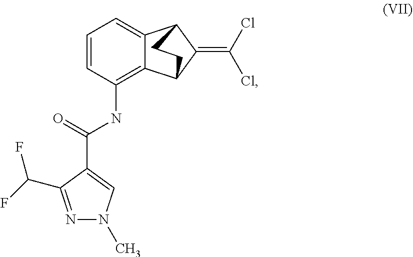

Preferred compositions comprising a compound of formula TX and

(B) a compound selected from the group consisting of
(B1) a strobilurin fungicide+TX, (B2) an azole fungicide+TX, (B3) a morpholine fungicide+TX, (B4) an anilinopyrimidine fungicide+TX, (B5) a fungicide selected from the group consisting of
anilazine (878)+TX, arsenates+TX, benalaxyl (56)+TX, benalaxyl-M+TX, benodanil (896)+TX, benomyl (62)+TX, benthiavalicarb+TX, benthiavalicarb-isopropyl (68)+TX, biphenyl (81)+TX, bitertanol (84)+TX, blasticidin-S (85)+TX, bordeaux mixture (87)+TX, boscalid (88)+TX, bupirimate (98)+TX, cadmium chloride+TX, captafol (113)+TX, captan (114)+TX, carbendazim (116)+TX, carbon disulfide (945)+TX, carboxin (120)+TX, carpropamid (122)+TX, cedar leaf oil+TX, chinomethionat (126)+TX, chlorine+TX, chloroneb (139)+TX, chlorothalonil (142)+TX, chlozolinate (149)+TX, cinnamaldehyde+TX, copper+TX, copper ammoniumcarbonate+TX, copper hydroxide (169)+TX, copper octanoate (170)+TX, copper oleate+TX, copper sulphate (87)+TX, cyazofamid (185)+TX, cycloheximide (1022)+TX, cymoxanil (200)+TX, dichlofluanid (230)+TX, dichlone (1052)+TX, dichloropropene (233)+TX, diclocymet (237)+TX, diclomezine (239)+TX, dicloran (240)+TX, diethofencarb (245)+TX, diflumetorim (253)+TX, dimethirimol (1082)+TX, dimethomorph (263)+TX, dinocap (270)+TX, dithianon (279)+TX, dodine (289)+TX, edifenphos (290)+TX, ethaboxam (304)+TX, ethirimol (1133)+TX, etridiazole (321)+TX, famoxadone (322)+TX, fenamidone (325)+TX, fenaminosulf (1144)+TX, fenamiphos (326)+TX, fenarimol (327)+TX, fenfuram (333)+TX, fenhexamid (334)+TX, fenoxanil (338)+TX, fenpiclonil (341)+TX, fentin acetate (347)+TX, fentin chloride+TX, fentin hydroxide (347)+TX, ferbam (350)+TX, ferimzone (351)+TX, fluazinam (363)+TX, fludioxonil (368)+TX, flusulfamide (394)+TX, flutolanil (396)+TX, folpet (400)+TX, formaldehyde (404)+TX, fosetyl-aluminium (407)+TX, fthalide (643)+TX, fuberidazole (419)+TX, furalaxyl (410)+TX, furametpyr (411)+TX, flyodin (1205)+TX, fuazatine (422)+TX, hexachlorobenzene (434)+TX, hymexazole+TX, iminoctadine (459)+TX, iodocarb (3-Iodo-2-propynyl butyl carbamate)+TX, iprobenfos (IBP) (469)+TX, iprodione (470)+TX, iprovalicarb (471)+TX, isoprothiolane (474)+TX, kasugamycin (483)+TX, mancozeb (496)+TX, maneb (497)+TX, manganous dimethyldithiocarbamate+TX, mefenoxam (Metalaxyl-M) (517)+TX, mepronil (510)+TX, mercuric chloride (511)+TX, mercury+TX, metalaxyl (516)+TX, methasulfocarb (528)+TX, metiram (546)+TX, metrafenone+TX, nabam (566)+TX, neem oil (hydrophobic extract)+TX, nuarimol (587)+TX, octhilinone (590)+TX, ofurace (592)+TX, oxadixyl (601)+TX, oxine copper (605)+TX, oxolinic acid (606)+TX, oxycarboxin (608)+TX, oxytetracycline (611)+TX, paclobutrazole (612)+TX, paraffin oil (628)+TX, paraformaldehyde+TX, pencycuron (620)+TX, pentachloronitrobenzene (716)+TX, pentachlorophenol (623)+TX, penthiopyrad+TX, perfurazoate+TX, phosphoric acid+TX, polyoxin (654)+TX, polyoxin D zinc salt (654)+TX, potassium bicarbonate+TX, probenazole (658)+TX, procymidone (660)+TX, propamocarb (668)+TX, propineb (676)+TX, proquinazid (682)+TX, prothiocarb (1361)+TX, pyrazophos (693)+TX, pyrifenox (703)+TX, pyroquilon (710)+TX, quinoxyfen (715)+TX, quintozene (PCNB) (716)+TX, silthiofam (729)+TX, sodium bicarbonate+TX, sodium diacetate+TX, sodium propionate+TX, streptomycin (744)+TX, sulphur (754)+TX, TCMTB+TX, tecloftalam+TX, tecnazene (TCNB) (767)+TX, thiabendazole (790)+TX, thifluzamide (796)+TX, thiophanate (1435)+TX, thiophanate-methyl (802)+TX, thiram (804)+TX, tolclofos-methyl (808)+TX, tolylfluanid (810)+TX, triazoxide (821)+TX, trichoderma harzianum (825)+TX, tricyclazole (828)+TX, triforine (838)+TX, triphenyltin hydroxide (347)+TX, validamycin (846)+TX, vinclozolin (849)+TX, zineb (855)+TX, ziram (856)+TX, zoxamide (857)+TX, 1,1-bis(4-chlorophenyl)-2-ethoxyethanol (IUPAC-Name) (910)+TX, 2+TX, 4-dichlorophenyl benzenesulfonate (IUPAC-/Chemical Abstracts-Name) (1059)+TX, 2-fluoro-N-methyl-N-1-naphthylacetamide (IUPAC-Name) (1295)+TX, 4-chlorophenyl phenyl sulfone (IUPAC-Name) (981)+TX,
a compound of formula B-5.1+TX

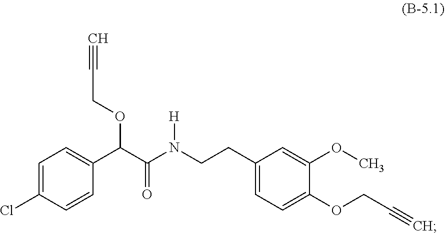

a compound of formula B-5.2+TX

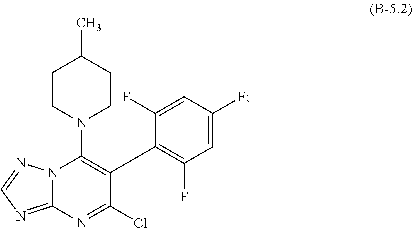

a compound of formula B-5.3+TX

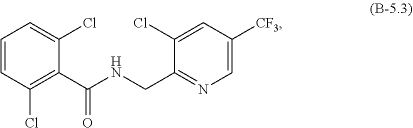

a compound of formula B-5.4+TX

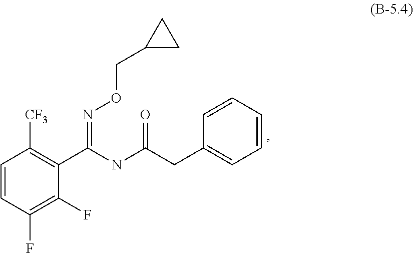

a compound of formula B-5.5+TX

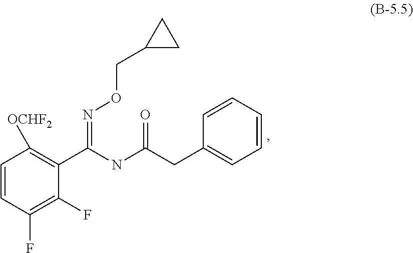

a compound of formula B-5.6+TX

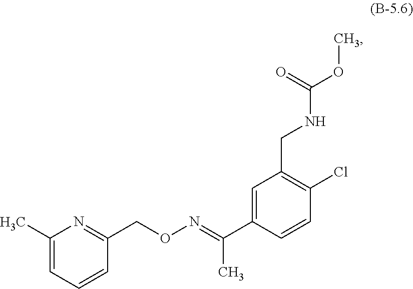

a compound of formula B-5.7+TX

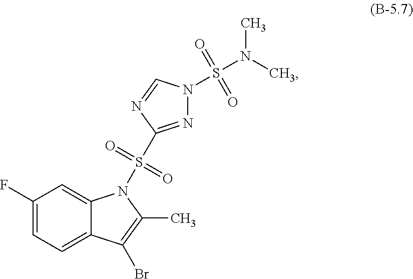

3-difluoromethyl-1-methyl-1H-pyrazole-4-carboxylic acid (2-bicyclopropyl-2-yl-phenyl)-amide (compound B-5.8)+TX, 3-difluoromethyl-1-methyl-1H-pyrazole-4-carboxylic acid (9-isopropyp-1,2,3,4-tetrahydro-1,4-methano-naphthalen-5-yl)-amide (compound B-5.9)+TX, 1,3-dimethyl-5-fluoro-1H-pyrazole-4-carboxylic acid [2-(1,3-dimethylbutyl)phenyl]-amide (compound B-5.10)+TX, 3-difluoromethyl-1-methyl-1H-pyrazole-4-carboxylic acid (3′,4′-dichloro-5-fluoro-1,1′-biphenyl-2-yl)-amide (compound B-5.11)+TX, N-{2-[3-chloro-5-(trifluoromethyl)pyridin-2-yl]ethyl}-2-(trifluoromethyl)benzamid (compound B-5.12)+TX, 3-difluoromethyl-1-methyl-1H-pyrazole-4-carboxylic acid N-[2-(1,1,2,2-tetrafluoroethoxy)phenyl]-amide (compound B-5.13)+TX, 3-difluoromethyl-1-methyl-1H-pyrazole-4-carboxylic acid N-[2-(1,1,2,3,3,3-hexafluoropropoxy)phenyl]-amide (compound B-5.14)+TX, 3-difluoromethyl-1-methyl-1H-pyrazole-4-carboxylic acid N-[2-(2-chloro-1,1,2-trifluoroethoxy)phenyl]-amide (compound B-5.15)+TX, 3-difluoromethyl-1-methyl-1H-pyrazole-4-carboxylic acid N-(4′-trifluoromethyl-biphen-2-yl)-amide (compound B-5.16)+TX, 3-difluoromethyl-1-methyl-1H-pyrazole-4-carboxylic acid N-(2′-trifluoromethyl-biphen-2-yl)-amide (compound B-5.17) and 3-difluoromethyl-1-methyl-1H-pyrazole-4-carboxylic acid N-(2′-trifluoromethyl-biphen-2-yl)-amide (compound B-5.18);
(B6) a plant-bioregulator selected from the group consisting of
acibenzolar-5-methyl (6)+TX, chlormequat chloride (137)+TX, ethephon (307)+TX, mepiquat chloride (509) and trinexapc-ethyl (841);
(B7) an insecticide selected from the group consisting of
abamectin (1)+TX, clothianidin (165)+TX, emamectin benzoate (291)+TX, imidacloprid (458)+TX, tefluthrin (769)+TX, thiamethoxam (792)+TX, a compound of formula B-7.1+TX

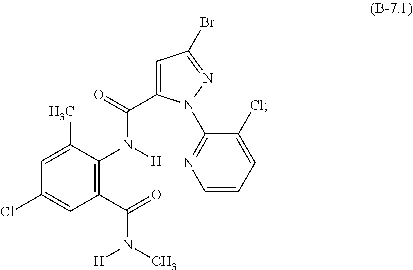

and a compound of formula B-7.2+TX;

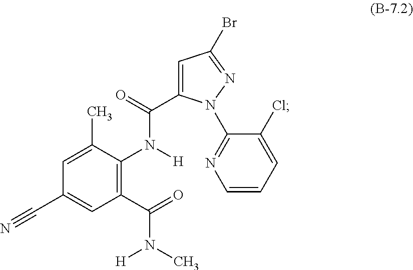

and (B8) glyphosate (419)+TX.

Examples of especially suitable mixtures selected from the following group P:

### Group P: Especially Suitable Mixtures According to the Invention:

a strobilurin fungicide selected from azoxystrobin (47)+TX, dimoxystrobin (226)+TX, fluoxastrobin (382)+TX, kresoxim-methyl (485)+TX, metominostrobin (551)+TX, orysastrobin+TX, picoxystrobin (647)+TX, pyraclostrobin (690); trifloxystrobin (832)+TX, a compound of formula B-1.1+TX

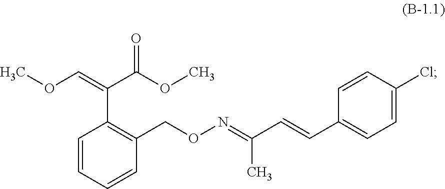

an azole fungicide selected from azaconazole (40)+TX, bromuconazole (96)+TX, cyproconazole (207)+TX, difenoconazole (247)+TX, diniconazole (267)+TX, diniconazole-M (267)+TX, epoxiconazole (298)+TX, fenbuconazole (329)+TX, fluquinconazole (385)+TX, flusilazole (393)+TX, flutriafol (397)+TX, hexaconazole (435)+TX, imazalil (449)+TX, imibenconazole (457)+TX, ipconazole (468)+TX, metconazole (525)+TX, myclobutanil (564)+TX, oxpoconazole (607)+TX, pefurazoate (618)+TX, penconazole (619)+TX, prochloraz (659)+TX, propiconazole (675)+TX, prothioconazole (685)+TX, simeconazole (731)+TX, tebuconazole (761)+TX, tetraconazole (778)+TX, triadimefon (814)+TX, triadimenol (815)+TX, triflumizole (834)+TX, triticonazole (842)+TX, diclobutrazol (1068)+TX, etaconazole (1129)+TX, furconazole (1198)+TX, furconazole-cis (1199) and quinconazole (1378);
a morpholine fungicide mixture selected from aldimorph+TX, dodemorph (288)+TX, fenpropimorph (344)+TX, tridemorph (830)+TX, fenpropidin (343)+TX, spiroxamine (740)+TX, piperalin (648) and a compound of formula B-3.1+TX

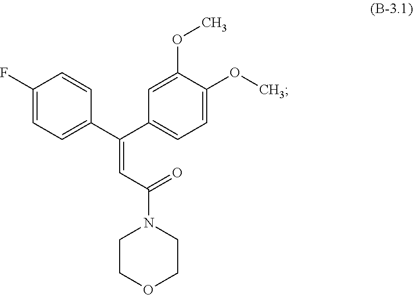

an anilino-pyrimidine fungicide selected from cyprodinil (208)+TX, mepanipyrim (508) and pyrimethanil (705);
a fungicide mixture selected from the group consisting of
anilazine (878)+TX, arsenates+TX, benalaxyl (56)+TX, benalaxyl-M+TX, benodanil (896)+TX, benomyl (62)+TX, benthiavalicarb+TX, benthiavalicarb-isopropyl (68)+TX, biphenyl (81)+TX, bitertanol (84)+TX, blasticidin-S (85)+TX, bordeaux mixture (87)+TX, boscalid (88)+TX, bupirimate (98)+TX, cadmium chloride+TX, captafol (113)+TX, captan (114)+TX, carbendazim (116)+TX, carbon disulfide (945)+TX, carboxin (120)+TX, carpropamid (122)+TX, cedar leaf oil+TX, chinomethionat (126)+TX, chlorine+TX, chloroneb (139)+TX, chlorothalonil (142)+TX, chlozolinate (149)+TX, cinnamaldehyde+TX, copper+TX, copper ammoniumcarbonate+TX, copper hydroxide (169)+TX, copper octanoate (170)+TX, copper oleate+TX, copper sulphate (87)+TX, cyazofamid (185)+TX, cycloheximide (1022)+TX, cymoxanil (200)+TX, dichlofluanid (230)+TX, dichlone (1052)+TX, dichloropropene (233)+TX, diclocymet (237)+TX, diclomezine (239)+TX, dicloran (240)+TX, diethofencarb (245)+TX, diflumetorim (253)+TX, dimethirimol (1082)+TX, dimethomorph (263)+TX, dinocap (270)+TX, dithianon (279)+TX, dodine (289)+TX, edifenphos (290)+TX, ethaboxam (304)+TX, ethirimol (1133)+TX, etridiazole (321)+TX, famoxadone (322)+TX, fenamidone (325)+TX, fenaminosulf (1144)+TX, fenamiphos (326)+TX, fenarimol (327)+TX, fenfuram (333)+TX, fenhexamid (334)+TX, fenoxanil (338)+TX, fenpiclonil (341)+TX, fentin acetate (347)+TX, fentin chloride+TX, fentin hydroxide (347)+TX, ferbam (350)+TX, ferimzone (351)+TX, fluazinam (363)+TX, fludioxonil (368)+TX, flusulfamide (394)+TX, flutolanil (396)+TX, folpet (400)+TX, formaldehyde (404)+TX, fosetyl-aluminium (407)+TX, fthalide (643)+TX, fuberidazole (419)+TX, furalaxyl (410)+TX, furametpyr (411)+TX, flyodin (1205)+TX, fuazatine (422)+TX, hexachlorobenzene (434)+TX, hymexazole+TX, iminoctadine (459)+TX, iodocarb (3-Iodo-2-propynyl butyl carbamate)+TX, iprobenfos (IBP) (469)+TX, iprodione (470)+TX, iprovalicarb (471)+TX, isoprothiolane (474)+TX, kasugamycin (483)+TX, mancozeb (496)+TX, maneb (497)+TX, manganous dimethyldithiocarbamate+TX, mefenoxam (Metalaxyl-M) (517)+TX, mepronil (510)+TX, mercuric chloride (511)+TX, mercury+TX, metalaxyl (516)+TX, methasulfocarb (528)+TX, metiram (546)+TX, metrafenone+TX, nabam (566)+TX, neem oil (hydrophobic extract)+TX, nuarimol (587)+TX, octhilinone (590)+TX, ofurace (592)+TX, oxadixyl (601)+TX, oxine copper (605)+TX, oxolinic acid (606)+TX, oxycarboxin (608)+TX, oxytetracycline (611)+TX, paclobutrazole (612)+TX, paraffin oil (628)+TX, paraformaldehyde+TX, pencycuron (620)+TX, pentachloronitrobenzene (716)+TX, pentachlorophenol (623)+TX, penthiopyrad+TX, perfurazoate+TX, phosphoric acid+TX, polyoxin (654)+TX, polyoxin D zinc salt (654)+TX, potassium bicarbonate+TX, probenazole (658)+TX, procymidone (660)+TX, propamocarb (668)+TX, propineb (676)+TX, proquinazid (682)+TX, prothiocarb (1361)+TX, pyrazophos (693)+TX, pyrifenox (703)+TX, pyroquilon (710)+TX, quinoxyfen (715)+TX, quintozene (PCNB) (716)+TX, silthiofam (729)+TX, sodium bicarbonate+TX, sodium diacetate+TX, sodium propionate+TX, streptomycin (744)+TX, sulphur (754)+TX, TCMTB+TX, tecloftalam+TX, tecnazene (TCNB) (767)+TX, thiabendazole (790)+TX, thifluzamide (796)+TX, thiophanate (1435)+TX, thiophanate-methyl (802)+TX, thiram (804)+TX, tolclofos-methyl (808)+TX, tolylfluanid (810)+TX, triazoxide (821)+TX, trichoderma harzianum (825)+TX, tricyclazole (828)+TX, triforine (838)+TX, triphenyltin hydroxide (347)+TX, validamycin (846)+TX, vinclozolin (849)+TX, zineb (855)+TX, ziram (856)+TX, zoxamide (857)+TX, 1+TX, 1-bis(4-chlorophenyl)-2-ethoxyethanol (IUPAC-Name) (910)+TX, 2+TX, 4-dichlorophenyl benzenesulfonate (IUPAC-/Chemical Abstracts-Name) (1059)+TX, 2-fluoro-N-methyl-N-1-naphthylacetamide (IUPAC-Name) (1295)+TX, 4-chlorophenyl phenyl sulfone (IUPAC-Name) (981)+TX,
a compound of formula B-5.1+TX, a compound of formula B-5.2+TX, a compound of formula B-5.3+TX, a compound of formula B-5.4+TX, a compound of formula B-5.5+TX, a compound of formula B-5.6+TX, a compound of formula B-5.7+TX, compound B-5.8+TX, compound B-5.9+TX, compound B-5.10+TX, compound B-5.11+TX, compound B-5.12+TX, compound B-5.13+TX, compound B-5.14+TX, compound B-5.15+TX, compound B-5.16+TX, compound B-5.17 and compound B-5.18;
a plant-bioregulator selected from the group consisting of
acibenzolar-5-methyl (6)+TX, chlormequat chloride (137)+TX, ethephon (307)+TX, mepiquat chloride (509) and trinexapc-ethyl (841);
an insecticide selected from the group consisting of
abamectin (1)+TX, clothianidin (165)+TX, emamectin benzoate (291)+TX, imidacloprid (458)+TX, tefluthrin (769)+TX, thiamethoxam (792)+TX, and glyphosate (419)+TX, a compound of formula V)+TX

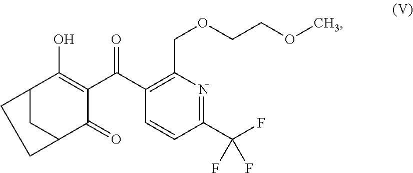

fomesafen+TX, and (B9) Isopyrazam+TX, Sedaxane+TX,
a compound of formula (VI)+TX

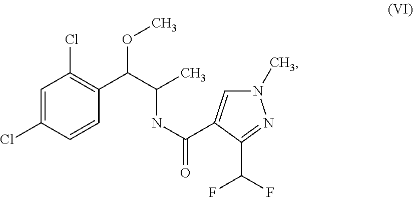

a compound of formula (VII)+TX

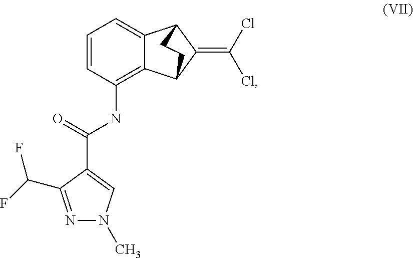

Further examples of especially suitable mixtures selected from the following group Q:

### Group Q: Especially Suitable Compositions According to the Invention:

a strobilurin fungicide selected from the group consisting of azoxystrobin+TX, dimoxystrobin+TX, fluoxastrobin+TX, kresoxim-methyl+TX, metominostrobin+TX, orysastrobin+TX, picoxystrobin+TX, pyraclostrobin; trifloxystrobin and a compound of formula B-1.1; an azole fungicide selected from the group consisting of azaconazole+TX, bromuconazole+TX, cyproconazole+TX, difenoconazole+TX, diniconazole+TX, diniconazole-M+TX, epoxiconazole+TX, fenbuconazole+TX, fluquinconazole+TX, flusilazole+TX, flutriafol+TX, hexaconazole+TX, imazalil+TX, imibenconazole+TX, ipconazole+TX, metconazole+TX, myclobutanil+TX, oxpoconazole+TX, pefurazoate+TX, penconazole+TX, prochloraz+TX, propiconazole+TX, prothioconazole+TX, simeconazole+TX, tebuconazole+TX, tetraconazole+TX, triadimefon+TX, triadimenol+TX, triflumizole+TX, triticonazole+TX, diclobutrazol+TX, etaconazole+TX, furconazole+TX, furconazole-cis and quinconazole;
a morpholine fungicide selected from the group consisting of aldimorph+TX, dodemorph+TX, fenpropimorph+TX, tridemorph+TX, fenpropidin+TX, spiroxamine+TX, piperalin and a compound of formula B-3.1;
an anilino-pyrimidine fungicide selected from the group consisting of cyprodinil+TX, mepanipyrim and pyrimethanil;
a fungicide selected from the group consisting of benalaxyl+TX, benalaxyl-M+TX, benomyl+TX, bitertanol+TX, boscalid+TX, captan+TX, carboxin+TX, carpropamid+TX, chlorothalonil+TX, copper+TX, cyazofamid+TX, cymoxanil+TX, diethofencarb+TX, dithianon+TX, famoxadone+TX, fenamidone+TX, fenhexamide+TX, fenoxycarb+TX, fenpiclonil+TX, fluazinam+TX, fludioxonil+TX, flutolanil+TX, folpet+TX, guazatine+TX, hymexazole+TX, iprodione+TX, lufenuron+TX, mancozeb+TX, metalaxyl+TX, mefenoxam+TX, metrafenone+TX, nuarimol+TX, paclobutrazol+TX, pencycuron+TX, penthiopyrad+TX, procymidone+TX, proquinazid+TX, pyroquilon+TX, quinoxyfen+TX, silthiofam+TX, sulfur+TX, thiabendazole+TX, thiram+TX, triazoxide+TX, tricyclazole+TX, a compound of formula B-5.1+TX, a compound of formula B-5.2+TX, a compound of formula B-5.3+TX, a compound of formula B-5.4+TX, a compound of formula B-5.5+TX, a compound of formula B-5.6+TX, a compound of formula B-5.7+TX, a compound of formula B-5.8+TX, a compound of formula B-5.9+TX, a compound of formula B-5.10 and a compound of formula B-5.12;
a plant-bioregulator selected from acibenzolar-5-methyl+TX, chlormequat chloride+TX, ethephon+TX, mepiquat chloride and trinexapc-ethyl;
an insecticide selected from abamectin+TX, emamectin benzoate+TX, tefluthrin+TX, thiamethoxam+TX, and glyphosate+TX, a compound of formula V

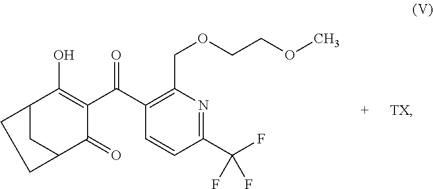

fomesafen+TX, and (B9) Isopyrazam+TX, Sedaxane+TX,
a compound of formula (VI)+TX

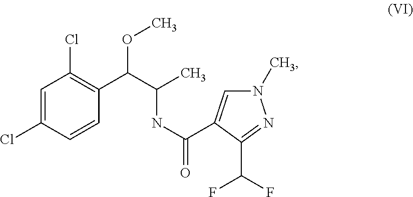

a compound of formula (VII)+TX

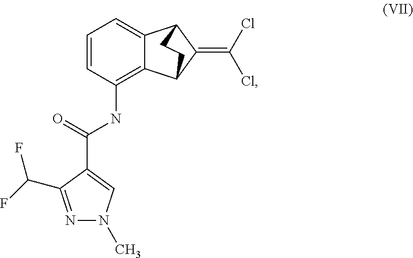

It has been found that the use of component (B) in combination with component TX surprisingly and substantially may enhance the effectiveness of the latter against fungi, and vice versa. Additionally, the method of the invention is effective against a wider spectrum of such fungi that can be combated with the active ingredients of this method, when used solely.

In general, the weight ratio of component TX to component (B) is from 2000:1 to 1:1000. A non-limiting example for such weight ratios is compound of formula I:compound of formula B-2 is 10:1. The weight ratio of component TX to component (B) is preferably from 100:1 to 1:100; more preferably from 20:1 to 1:50.

The active ingredient mixture of component TX to component (B) comprises compounds of formula I and a further, other biocidally active ingredients or compositions or if desired, a solid or liquid adjuvant preferably in a mixing ratio of from 1000:1 to 1:1000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are understood to include, on the one hand, ratios by weight and also, on other hand, molar ratios.

It has been found, surprisingly, that certain weight ratios of component TX to component (B) are able to give rise to synergistic activity. Therefore, a further aspect of the invention are compositions, wherein component TX and component (B) are present in the composition in amounts producing a synergistic effect. This synergistic activity is apparent from the fact that the fungicidal activity of the composition comprising component TX and component (B) is greater than the sum of the fungicidal activities of component TX and of component (B). This synergistic activity extends the range of action of component TX and component (B) in two ways. Firstly, the rates of application of component TX and component (B) are lowered whilst the action remains equally good, meaning that the active ingredient mixture still achieves a high degree of phytopathogen control even where the two individual components have become totally ineffective in such a low application rate range. Secondly, there is a substantial broadening of the spectrum of phytopathogens that can be controlled.

A synergistic effect exists whenever the action of an active ingredient combination is greater than the sum of the actions of the individual components. The action to be expected E for a given active ingredient combination obeys the so-called COLBY formula and can be calculated as follows (COLBY, S. R. “Calculating synergistic and antagonistic responses of herbicide combination”. Weeds, Vol. 15, pages 20-22; 1967):

ppm=milligrams of active ingredient (=a.i.) per liter of spray mixture
X=% action by active ingredient A) using p ppm of active ingredient
Y=% action by active ingredient B) using q ppm of active ingredient.

According to COLBY, the expected (additive) action of active ingredients A)+B) using p+q ppm of active ingredient is

$$E = X + Y - \frac{X \cdot Y}{100}$$

If the action actually observed (O) is greater than the expected action (E), then the action of the combination is super-additive, i.e. there is a synergistic effect. In mathematical terms, synergism corresponds to a positive value for the difference of (O-E). In the case of purely complementary addition of activities (expected activity), said difference (O-E) is zero. A negative value of said difference (O-E) signals a loss of activity compared to the expected activity.

However, besides the actual synergistic action with respect to fungicidal activity, the compositions according to the invention can also have further surprising advantageous properties. Examples of such advantageous properties that may be mentioned are: more advantageous degradability; improved toxicological and/or ecotoxicological behaviour; or improved characteristics of the useful plants including: emergence, crop yields, more developed root system, tillering increase, increase in plant height, bigger leaf blade, less dead basal leaves, stronger tillers, greener leaf colour, less fertilizers needed, less seeds needed, more productive tillers, earlier flowering, early grain maturity, less plant verse (lodging), increased shoot growth, improved plant vigor, and early germination.

Some compositions according to the invention have a systemic action and can be used as foliar, soil and seed treatment fungicides.

With the compositions according to the invention it is possible to inhibit or destroy the phytopathogenic microorganisms which occur in plants or in parts of plants (fruit, blossoms, leaves, stems, tubers, roots) in different useful plants, while at the same time the parts of plants which grow later are also protected from attack by phytopathogenic microorganisms. The compositions according to the invention can be applied to the phytopathogenic microorganisms, the useful plants, the locus thereof, the propagation material thereof, storage goods or technical materials threatened by microorganism attack.

The compositions according to the invention may be applied before or after infection of the useful plants, the propagation material thereof, storage goods or technical materials by the microorganisms.

A further aspect of the present invention is a method of controlling diseases on useful plants or on propagation material thereof caused by phytopathogens, which comprises applying to the useful plants, the locus thereof or propagation material thereof a composition according to the invention. Preferred is a method, which comprises applying to the useful plants or to the locus thereof a composition according to the invention, more preferably to the useful plants. Further preferred is a method, which comprises applying to the propagation material of the useful plants a composition according to the invention.

The components (B) are known. Where the components (B) are included in “The Pesticide Manual” [The Pesticide Manual—A World Compendium; Thirteenth Edition; Editor: C. D. S. Tomlin; The British Crop Protection Council], they are described therein under the entry number given in round brackets hereinabove for the particular component (B); for example, the compound “abamectin” is described under entry number (1). Most of the components (B) are referred to hereinabove by a so-called “common name”, the relevant “ISO common name” or another “common name” being used in individual cases. If the designation is not a “common name”, the nature of the designation used instead is given in round brackets for the particular component (B); in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed.

The following components B) are registered under a CAS-Reg. No.

aldimorph (CAS 91315-15-0); arsenates (CAS 1327-53-3); benalaxyl-M (CAS 98243-83-5); benthiavalicarb (CAS 413615-35-7); cadmium chloride (CAS 10108-64-2); cedar leaf oil (CAS 8007-20-3); chlorine (CAS 7782-50-5); cinnamaldehyde (CAS: 104-55-2); copper ammoniumcarbonate (CAS 33113-08-5); copper oleate (CAS 1120-44-1); iodocarb (3-Iodo-2-propynyl butyl carbamate) (CAS 55406-53-6); hymexazole (CAS 10004-44-1); manganous dimethyldithiocarbamate (CAS 15339-36-3); mercury (CAS 7487-94-7; 21908-53-2; 7546-30-7); metrafenone (CAS 220899-03-6); neem oil (hydrophobic extract) (CAS 8002-65-1); orysastrobin CAS 248593-16-0); paraformaldehyde (CAS 30525-89-4); penthiopyrad (CAS 183675-82-3); phosphoric acid (CAS 7664-38-2); potassium bicarbonate (CAS 298-14-6); sodium bicarbonate (CAS 144-55-8); sodium diacetate (CAS 127-09-3); sodium propionate (CAS 137-40-6); TCMTB (CAS 21564-17-0); and tolyfluanid (CAS 731-27-1). Compound B-1.1 (“enestrobin”) is described in EP-0-936-213; compound B-3.1 (“flumorph”) in U.S. Pat. No. 6,020,332, CN-1-167-568, CN-1-155-977 and in EP-0-860-438; compound B-5.1 (“mandipropamid”) in WO 01/87822; compound B-5.2 in WO 98/46607; compound B-5.3 (“fluopicolide”) in WO 99/42447; compound B-5.4 (“cyflufenamid”) in WO 96/19442; compound B-5.5 in WO 99/14187; compound B-5.6 (“pyribencarb”) is registered under CAS-Reg. No. 325156-49-8; compound B-5.7 (“amisulbrom” or “ambromdole”) is registered under CAS-Reg. No. 348635-87-0; compound B-5.8 (3-difluoromethyl-1-methyl-1H-pyrazole-4-carboxylic acid (2-bicyclopropyl-2-yl-phenyl)-amide) is described in WO 03/74491; compound B-5.9 (3-difluoromethyl-1-methyl-1H-pyrazole-4-carboxylic acid (9-isopropyp-1,2,3,4-tetrahydro-1,4-methano-naphthalen-5-yl)-amide) is described in WO 04/35589 and in WO 06/37632; compound B-5.10 (1,3-dimethyl-5-fluoro-1H-pyrazole-4-carboxylic acid [2-(1,3-dimethylbutyl)phenyl]-amide) is described in WO 03/10149; compound B-5.11 (3-difluoromethyl-1-methyl-1H-pyrazole-4-carboxylic acid (3′,4′-dichloro-5-fluoro-1,1′-biphenyl-2-yl)-amide; “bixafen”) is registered under CAS-Reg. No.: 581809-46-3 and described in WO 03/70705; compound B-5.12 (N-{2-[3-Chloro-5-(trifluoromethyl)pyridin-2-yl]ethyl}-2-(trifluoromethyl)benzamid; “fluopyram”) is registered under CAS-Reg. No: 658066-35-4 and described in WO 04/16088; compounds B-5.13, B-5.14 and B-5.15 are described in WO 2007/17450; compounds B-5.16, B-5.17 and B-5.18 are described in WO 2006/120219; The compounds of formula IV are for example described in WO 04/067528, WO 2005/085234, WO 2006/111341, WO 03/015519, WO 2007/020050, WO 2006/040113, and WO 2007/093402. The compound of formula V is described in WO 2001/094339. Isopyraxam (3-(difluoromethyl)-1-methyl-N-[1,2,3,4-tetrahydro-9-(1-methylethyl)-1,4-methanonaphthalen-5-yl]-1H-pyrazole-4-carboxamide) is described in WO 2004/035589, in WO 2006/037632 and in EP1556385B1 and is registered under the CAS-Reg. 881685-58-1. Sedaxane (N-[2-[1,1′-bicyclopropyl]-2-ylphenyl]-3-(difluoromethyl)-1-methyl-1H-pyrazole-4-carboxamide) is described in WO 2003/074491 and is registered under the CAS-Reg. 874967-67-6; The compound of formula (VI) is described in WO 2008/014870; and the compounds of formula (VII) is described in WO 2007/048556. Fomesafen is registered under the CAS-Reg. No. 72178-02-0.

Throughout this document the expression “composition” stands for the various mixtures or combinations of components TX and (B), for example in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the components TX and (B) is not essential for working the present invention.

The compositions according to the invention may also comprise more than one of the active components (B), if, for example, a broadening of the spectrum of disease control is desired. For instance, it may be advantageous in the agricultural practice to combine two or three components (B) with component TX. An example is a composition comprising a compound of formula (I), azoxystrobin and cyproconazole.

The compounds of formula (I), or a pharmaceutical salt thereof, described above may also have an advantageous spectrum of activity for the treatment and/or prevention of microbial infection in an animal. “Animal” can be any animal, for example, insect, mammal, reptile, fish, amphibian, preferably mammal, most preferably human. “Treatment” means the use on an animal which has microbial infection in order to reduce or slow or stop the increase or spread of the infection, or to reduce the infection or to cure the infection. “Prevention” means the use on an animal which has no apparent signs of microbial infection in order to prevent any future infection, or to reduce or slow the increase or spread of any future infection. According to the present invention there is provided the use of a compound of formula (I) in the manufacture of a medicament for use in the treatment and/or prevention of microbial infection in an animal. There is also provided the use of a compound of formula (I) as a pharmaceutical agent. There is also provided the use of a compound of formula (I) as an antimicrobial agent in the treatment of an animal. According to the present invention there is also provided a pharmaceutical composition comprising as an active ingredient a compound of formula (I), or a pharmaceutically acceptable salt thereof, and a pharmaceutically acceptable diluent or carrier. This composition can be used for the treatment and/or prevention of antimicrobial infection in an animal. This pharmaceutical composition can be in a form suitable for oral administration, such as tablet, lozenges, hard capsules, aqueous suspensions, oily suspensions, emulsions dispersible powders, dispersible granules, syrups and elixirs. Alternatively this pharmaceutical composition can be in a form suitable for topical application, such as a spray, a cream or lotion. Alternatively this pharmaceutical composition can be in a form suitable for parenteral administration, for example injection. Alternatively this pharmaceutical composition can be in inhalable form, such as an aerosol spray.

The compounds of formula (I) may be effective against various microbial species able to cause a microbial infection in an animal. Examples of such microbial species are those causing Aspergillosis such as Aspergillus fumigatus, A. flavus, A. terrus, A. nidulans and A. niger; those causing Blastomycosis such as Blastomyces dermatitidis; those causing Candidiasis such as Candida albicans, C. glabrata, C. tropicalis, C. parapsilosis, C. krusei and C. lusitaniae; those causing Coccidioidomycosis such as Coccidioides immitis; those causing Cryptococcosis such as Cryptococcus neoformans; those causing Histoplasmosis such as Histoplasma capsulatum and those causing Zygomycosis such as Absidia corymbifera, Rhizomucor pusillus and Rhizopus arrhizus. Further examples are Fusarium Spp such as Fusarium oxysporum and Fusarium solani and Scedosporium Spp such as Scedosporium apiospermum and Scedosporium prolificans. Still further examples are Microsporum Spp, Trichophyton Spp, Epidermophyton Spp, Mucor Spp, Sporothorix Spp, Phialophora Spp, Cladosporium Spp, Petriellidium spp, Paracoccidioides Spp and Histoplasma Spp.

The following non-limiting examples illustrate the above-described invention in greater detail without limiting it.

### PREPARATORY EXAMPLES

### Example P1

### Preparation of 2-(6-benzyl-pyridin-2-yl)-4-chloro-thieno[2,3-d]pyrimidine

a) Preparation of 2-(6-bromo-pyridin-2-yl)-3H-thieno[2,3-d]pyrimidin-4-one:

To a solution of 2-amino-thiophene-3-carboxylic acid amide (500 mg, 3.52 mmol) in ethanol (20 ml) was successively added sodium ethanolate (141 mg, 8.79 mmol) and 6-bromo-pyridine-2-carboxylic acid methyl ester (912 mg, 4.22 mmol). The solution was heated under reflux for 3 hours, cooled to ambient temperature and acidified with 1N hydrogen chloride. The precipitate that formed was then filtered and thoroughly washed with water to afford 2-(6-bromo-pyridin-2-yl)-3H-thieno[2,3-d]pyrimidin-4-one as a yellow solid. ¹H NMR (400 MHz, CDCl₃) δ ppm 7.36 (d, J=5.87 Hz, 1H), 7.58 (d, J=5.50 Hz, 1H), 7.66 (dd, J=8.80, 0.73 Hz, 1H), 7.77 (t, J=8.10 Hz, 1H), 8.45 (dd, J=7.70, 0.73 Hz, 1H), 10.79 (br. s, 1H)
b) Preparation of 2-(6-benzyl-pyridin-2-yl)-3H-thieno[2,3-d]pyrimidin-4-one:

To a solution of 2-(6-bromo-pyridin-2-yl)-3H-thieno[2,3-d]pyrimidin-4-one (9.50 g, 30.8 mmol) in tetrahydrofuran (30 ml) under argon atmosphere was successively added tetrakis(triphenylphosphine)-palladium(0) (3.56 g, 3.08 mmol) and benzylzinc bromide (185 ml, 0.5M in tetrahydrofurane, 92.5 mmol). The solution was heated under reflux for 5 hours, after which the reaction mixture was cooled to ambient temperature and concentrated. The concentrated solution was diluted with ethyl acetate, water was added and the resulting precipitate was filtered. The precipitate was suspended in ethyl acetate and treated with a basic solution of ethylenediaminetetraacetic acid until it completely dissolved. The aqueous phase was extracted with ethyl acetate and the combined organic phases were dried over sodium sulfate and concentrated under reduced pressure. The 2-(6-benzyl-pyridin-2-yl)-3H-thieno[2,3-d]pyrimidin-4-one was obtained in form of a yellow solid. ¹H NMR (400 MHz, CDCl₃) δ ppm 4.23 (s, 2H), 7.22-7.39 (m, 7H), 7.57 (d, J=5.9 Hz, 1H), 7.79 (t, J=7.7 Hz, 1H), 8.31 (dd, J=7.7, 1.0 Hz, 1H), 11.05 (br. s, 1H).
c) A mixture of 2-(6-benzyl-pyridin-2-yl)-3H-thieno[2,3-d]pyrimidin-4-one (84 mg, 0.263 mmol) and phosphorus oxychloride (202 mg, 1.32 mmol) was heated under microwaves irradiation at 120° C. for 10 min. The reaction solution was poured in a saturated sodium bicarbonate solution and extracted with ethyl acetate. The organic layers were washed with sodium bicarbonate and brine, dried over magnesium sulfate and concentrated under reduced pressure. The resulting oil was purified by flash chromatography, using 50 to 100% dichloromethane in heptanes, to afford 2-(6-benzyl-pyridin-2-yl)-4-chloro-thieno[2,3-d]pyrimidine as a light orange gum. ¹H NMR (400 MHz, CDCl₃) δ ppm 4.48 (s, 2H), 7.15 (dd, J=7.7, 1.1 Hz, 1H), 7.26 (m, 1H), 7.33 (m, 4H), 7.49 (dd, J=5.9, 1.0 Hz, 0H), 7.68 (d, J=6.2 Hz, 1H), 7.78 (t, J=7.9 Hz, 1H), 8.43 (dd, J=7.7, 1.0 Hz, 1H).

### Example P2

### Preparation of: 2-(6-benzyl-pyridin-2-yl)-4-methoxy-thieno[2,3-d]pyrimidine

a) A mixture of 2-(6-benzyl-pyridin-2-yl)-3H-thieno[2,3-d]pyrimidin-4-one (75 mg, 0.24 mmol) and phosphorus oxychloride (180 mg, 1.17 mmol) was heated under microwaves irradiation at 120° C. for 10 min. The resulting brown liquid was concentrated under reduced pressure. To the resulting brown oil was slowly added methanol (0.5 ml) and then a solution on sodium methoxide (30% wt. in methanol, 0.44 ml, 2.4 mmol). The reaction mixture was stirred for 1 h at ambient temperature. Ethyl acetate was added to the reaction mixture and the organic phase was washed using a saturated solution of sodium bicarbonate. The aqueous phase was extracted with ethyl acetate and the combined organic phases were dried over sodium sulfate, and concentrated under reduced pressure. The resulting oil was purified by flash chromatography, using 25% ethyl acetate in cyclohexane containing 1% of triethylamine, to afford 2-(6-benzyl-pyridin-2-yl)-4-methoxy-thieno[2,3-d]pyrimidine as a colorless oil. ¹H NMR (400 MHz, CDCl₃) δ ppm 4.27 (s, 3H), 4.43 (s, 2H), 7.08 (d, J=7.3 Hz, 1H), 7.23-7.27 (m, 1H), 7.32-7.36 (m, 4H), 7.41 (dd, J=5.9, 1.0 Hz, 1 H), 7.45 (dd, J=5.9, 1.0 Hz, 1H), 7.72 (t, J=7.9 Hz, 1H), 8.40 (dd, J=7.7, 1.0 Hz, 1 H).

### Example P3

### Preparation of 2-(6-benzyl-pyridin-2-yl)-4-ethoxy-thieno[2,3-d]pyrimidine

a) A mixture of 2-(6-benzyl-pyridin-2-yl)-3H-thieno[2,3-d]pyrimidin-4-one (50 mg, 0.16 mmol) and phosphorus oxychloride (168 mg, 1.10 mmol) was heated under microwaves irradiation at 120° C. for 10 min. The resulting brown liquid was concentrated under reduced pressure. To the resulting brown oil was slowly added ethanol (0.5 ml) and then a solution on sodium ethoxide (21% wt. in ethanol, 0.58 ml, 1.57 mmol). The reaction mixture was stirred for 1 hour at ambient temperature. Ethyl acetate was added to the reaction mixture and the organic phase was washed using a saturated solution of sodium bicarbonate. The aqueous phase was extracted with ethyl acetate and the combined organic phases were dried over sodium sulfate and concentrated under reduced pressure. The resulting oil was purified by flash chromatography, using 25% ethyl acetate in cyclohexane containing 1% of triethylamine, to afford 2-(6-benzyl-pyridin-2-yl)-4-ethoxy-thieno[2,3-d]pyrimidine as a white solid. ¹H NMR (400 MHz, CDCl₃) δ ppm 1.55 (t, J=7.2 Hz, 3H), 4.43 (s, 2H), 4.76 (q, J=7.3 Hz, 2H), 7.08 (dd, J=7.7, 1.0 Hz, 1H), 7.22-7.27 (m, 1H), 7.29-7.36 (m, 4H), 7.41 (dd, J=5.9, 1.0 Hz, 1H), 7.43 (dd, J=5.9, 1.0 Hz, 1H), 7.71 (t, J=7.7 Hz, 1H), 8.36 (d, J=7.7 Hz, 1H).

### Example P4

### Preparation of 2-(6-benzyl-pyridin-2-yl)-thieno[2,3-d]pyrimidine

a) A mixture of 2-(6-benzyl-pyridin-2-yl)-3H-thieno[2,3-d]pyrimidin-4-one (51 mg, 0.16 mmol) and phosphorus oxychloride (168 mg, 1.10 mmol) was heated under microwaves irradiation for 10 min at 120° C. The reaction mixture was quenched by slowly pouring into a saturated solution of bicarbonate and extracted with ethyl acetate. The combined organic layers were washed with a solution of saturate sodium bicarbonate, dried over sodium sulfate, and concentrated under reduced pressure. The resulting brown oil was dissolved in a mixture of methanol (2.5 ml) and triethylamine (134 μl) under an atmosphere of argon, followed by the addition of palladium on carbon (17 mg, 0.016 mmol). The mixture was transferred under an atmosphere of hydrogen and stirred for 48 hours. The reaction mixture was then filtered through a plug of celite and concentrated under reduced pressure. The resulting oil was purified by flash chromatography, using 50% ethyl acetate in cyclohexane containing 1% of triethylamine, to afford 2-(6-benzyl-pyridin-2-yl)-thieno[2,3-d]pyrimidine as a yellow solid. ¹H NMR (400 MHz, CDCl₃) δ ppm 4.45 (s, 2 H), 7.10 (dd, J=7.7, 1.0 Hz, 1H), 7.21-7.28 (m, 1H), 7.30-7.35 (m, 4H), 7.42 (d, J=5.9 Hz, 1H), 7.62 (d, J=6.2 Hz, 1H), 7.74 (t, J=7.9 Hz, 1H), 8.44 (d, J=7.3 Hz, 1 H), 9.36 (s, 1H).

### Example P5

### Preparation of 2-(5-methyl-6-phenyl-pyridin-2-yl)-pyrido[2,3-d]pyrimidine

a) Preparation of 3-methyl-2-phenyl-pyridine:

To a stirred solution of 2-bromo-3-methylpyridine (30 g, 174 mmol) in dimethoxyethane (1.3 l) was added in one portion phenylboronic acid (42.5 g, 349 mmol) at ambient temperature, followed by an aqueous solution of sodium carbonate (3 M in water, 233 ml, 698 mmol). The mixture was degassed with argon for about 30 minutes, after which [1,1′-bis(diphenylphosphino)ferrocene]dichloropalladium(II) complex with dichloromethane (4.3 g, 5.0 mmol) was added under argon atmosphere. The reaction was stirred at 95° C. for 2 hours. The crude mixture was diluted with ethyl acetate and water and the organic layer was decanted. It was washed once with an aqueous solution of sodium hydroxide (0.5 M) and once with brine. The organic layer was collected, dried with sodium sulphate and concentrated in vacuo. The crude mixture was purified by flash chromatography on silica gel (eluent: ethyl acetate/cyclohexane 1:3). The title compound was obtained as a pale orange oil. ¹H-NMR (CDCl₃): δ=2.37 (s, 3H), 7.19 (dd, 1H), 7.37-7.41 (m, 2H), 7.42-7.49 (dd, 1H), 7.52-7.56 (m, 2H), 7.60 (d, 1H), 8.55 (d, 1H).
b) Preparation of 3-methyl-2-phenyl-pyridine 1-oxide:

To a stirred solution of 3-methyl-2-phenyl-pyridine (26.9 g, 159 mmol) in dichloromethane (220 ml) under nitrogen atmosphere was added m-chloroperbenzoic acid (70% pure, 78.4 g, 318 mmol) in small portions, at 0° C. The mixture was stirred for 18 hours at ambient temperature. It was then cooled to 0° C. and an aqueous solution of sodium hydroxide (2 M) was added slowly (exothermic reaction!) until a basic pH was reached. To this mixture was then added a saturated aqueous solution of sodium thiosulphate slowly at 0° C. (highly exothermic reaction!). The biphasic solution was stirred for an additional 30 minutes after which the organic layer was decanted, washed with an aqueous solution of sodium hydroxide (1 M), decanted, dried over sodium sulphate and concentrated in vacuo. The crude compound was obtained as a white solid. ¹H-NMR (CDCl₃): δ=2.13 (s, 3H), 7.15-7.22 (m, 2H), 7.47 (d, 2H), 7.43-7.49 (m, 1H), 7.51-7.57 (m, 2H), 8.27 (d, 1H).
c) Preparation of 5-methyl-6-phenyl-1H-pyridin-2-one:

A solution of 3-methyl-2-phenyl-pyridine 1-oxide (12 g, 65 mmol) in acetic anhydride (120 ml) was equally partitioned in four microwave vials and sealed. The vials were irradiated for 45 minutes in a microwave oven at a temperature of 175° C. The crude mixture was concentrated in vacuo. The crude was taken up in ethyl acetate (100 ml) and an aqueous solution of lithium hydroxide (1 M) was added until ph˜9 was reached. The mixture was vigorously stirred for an hour after which the organic layer was decanted. The aqueous layer was extracted three times with ethyl acetate. The organic layers were collected, dried over magnesium sulphate and concentrated in vacuo. The crude mixture was purified by flash chromatography on silica gel (eluent gradient: pure dichloromethane to 6% methanol in dichloromethane). The title compound was obtained as a white solid. ¹H NMR (CDCl₃)=2.10 (s, 3H), 6.51 (d, 1H), 7.36 (d, 1H), 7.41-7.46 (m, 2H), 7.48-7.52 (m, 3H), 9.73 (s, 1H).
d) Preparation of 6-bromo-3-methyl-2-phenyl-pyridine:

To a solution of 5-methyl-6-phenyl-1H-pyridin-2-one (1.6 g, 8.6 mmol) in toluene (35 ml) was added in one portion phosphorus oxybromide (5.0 g, 17.3 mmol). The mixture was refluxed for 2 hours, and then cooled to 0° C., covered with ethyl acetate and quenched with an aqueous solution of sodium hydroxide (2 M) at 0° C. The organic layer was decanted, dried and concentrated. The crude mixture was filtered over a pad of silica gel with a mixture of 25% ethyl acetate in cyclohexane. The title compound was obtained as a colourless oil. ¹H NMR (CDCl₃)=2.34 (s, 3H), 7.39 (d, 1H), 7.40-7.48 (m, 5H), 7.53 (d, 1H).
e) Preparation of 3-methyl-2-phenyl-6-tributylstannanyl-pyridine:

In a dry flask, under argon, a solution of n-butyl lithium in tetrahydrofuran (1.5 M, 17 mL, 25.7 mmol) was added dropwise to a solution of 6-bromo-3-methyl-2-phenyl-pyridine (5.8 g, 23.4 mmol) in anhydrous tetrahydrofuran (100 mL), at −78° C. The solution was stirred at that temperature for 45 minutes, after which tributyltin chloride (6.4 mL, 23.4 mmol) was added dropwise, at −78° C. The solution was allowed to warm up to ambient temperature over an hour, before which a saturated aqueous solution of ammonium chloride was added. The organic layer was decanted. The aqueous layer was further extracted twice with ethyl acetate. The organic layers were collected, dried over magnesium sulphate and concentrated in vacuo. The title compound was obtained as a pale yellow oil. ¹H NMR (CDCl₃): 0.92 (m, 9H), 1.14 (m, 6H), 1.48 (m, 6H), 1.60 (m, 6H), 7.28 (d, 1H), 7.47-7.50 (m, 2H), 7.52-7.58 (m, 2H), 7.61 (m, 2H).
f) Preparation of 1H-pyrido[2,3-d]pyrimidine-2,4-dione:

A mixture of 2-aminopicotinic acid (1.4 g, 10 mmol) and urea (3.7 g, 61 mmol) was heated to 190° C. for 3 hours, after which it was cooled to 100° C., and water, then aqueous hydrochloric acid (1 M) were added, until an acidic pH was reached. The mixture was refluxed for an hour before being cooled to ambient temperature. The resulting solid was filtered and dried in vacuo. ¹H NMR (DMSO-d₆): 7.24 (dd, 1H), 8.26 (dd, 1H), 8.59 (dd, 1H), 10.95 (s, 1H), 11.70 (s, 1H).
g) Preparation of 2,4-dichloro-pyrido[2,3-d]pyrimidine:

A mixture of 1H-pyrido[2,3-d]pyrimidine-2,4-dione (1.4 g, 8.5 mmol) and N,N-diethylaniline (1.4 mL, 8.5 mmol) in phosphorus oxychloride (8 ml) was refluxed for 3 hours. It was then concentrated in vacuo and the residue was carefully poured on an ice-cold saturated aqueous solution of sodium bicarbonate. The suspension was diluted with ethyl acetate and the organic layer was decanted, dried over magnesium sulphate and concentrated in vacuo. The crude oil was purified by flash chromatography on silica gel (eluent gradient: 0% to 60% ethyl acetate in cyclohexane). The title compound was obtained as a white solid. ¹H NMR (CDCl₃): 7.72 (dd, 1H), 8.64 (dd, 1H), 9.43 (dd, 1H).
h) Preparation of 2-chloro-pyrido[2,3-d]pyrimidine:

To a degassed, stirred solution of 2,4-dichloro-pyrido[2,3-d]pyrimidine (150 mg, 0.75 mmol) and tributyltin hydride (0.22 ml, 0.83 mmol) in toluene (10 ml) was added tetrakis(triphenylphosphine)palladium(0) (87 mg, 75 mmol). The solution, kept under argon, was immersed in a preheated oil bath (100° C.) and stirred at that temperature for 3 hours. The mixture was cooled down to ambient temperature, concentrated in vacuo to about 2 ml and diluted with acetonitrile. It was extracted 3 times with hexane. The acetonitrile layer was concentrated in vacuo and the residue was purified by flash chromatography on silica gel (eluent gradient: 0% to 6% methanol in dichloromethane). The title compound was obtained as a white solid. ¹H NMR (CDCl₃): 7.69 (dd, 1H), 8.40 (dd, 1H), 9.33 (dd, 1H), 9.42 (s, 1H).
i) To a degassed, stirred solution of 3-methyl-2-phenyl-6-tributylstannanyl-pyridine (334 mg, 0.58 mmol), 2-chloro-pyrido[2,3-d]pyrimidine (77 mg, 0.47 mmol) and lithium chloride (55 mg, 1.3 mmol) in anhydrous N,N-dimethylformamide (3 ml) in a supelco vial, was added tetrakis(triphenylphosphine)palladium(0) (54 mg, 47 μmol). The vial was sealed and heated for 18 hours to 100° C. The crude mixture was then diluted with acetonitrile and washed 3 times with hexane. The acetonitrile layer was concentrated in vacuo and taken up in ethyl acetate. It was washed 3 times with water, dried over magnesium sulphate and concentrated. It was redissolved in dichloromethane and stirred vigorously with a saturated aqueous solution of sodium bicarbonate for 2 hours. The organic layer was decanted, dried and concentrated. The crude thus obtained was purified by flash chromatography on silica gel (eluent gradient: 90% to 100% ethyl acetate in cyclohexane). The title compound was obtained as a white solid. m.p.: 199-201° C. ¹H NMR (CDCl₃): 2.43 (s, 3H), 7.36-7.40 (m, 1H), 7.43-7.48 (m, 2H), 7.60-7.63 (m, 3H), 7.70 (d, 1H), 8.45 (dd, 1H), 8.72 (app. d, 1H), 9.30 (dd, 1H), 9.68 (s, 1H).

### Example P6

### Preparation of 2-(6-Benzyl-pyridin-2-yl)-thieno[3,2-d]pyrimidine

a) 2-(6-benzyl-2-pyridyl)-4-chloro-thieno[3,2-d]pyrimidine was synthesized according to the procedure described above for the synthesis of 2-(6-benzyl-2-pyridyl)-4-chloro-thieno[2,3-d]pyrimidine as in Example P1, using 3-amino-thiophene-2-carboxylic acid amide as a starting material.
b) To a solution of 2-(6-benzyl-2-pyridyl)-4-chloro-thieno[3,2-d]pyrimidine (140 mg, 0.44 mmol) in a 2:1 mixture of THF:MeOH (0.3M), was added triethylamine until pH=4-5. The solution was degassed (3 cycles of vacuum/Argon) and 5% Pd/C (140 mg) was added. The resulting mixture was stirred overnight under an atmosphere of hydrogen. The reaction mixture was then filtered through a plug of celite and the precipitate washed with hot methanol. The filtrate was concentrated under reduced pressure and the residue was purified by flash chromatography, using 10% ethyl acetate in dichloromethane, to afford 2-(6-benzyl-2-pyridyl)thieno[3,2-d]pyrimidine as a light yellow solid. ¹H NMR (400 MHz, CDCl₃-d) 8 ppm 4.45 (s, 2H) 7.10 (d, J=7.7 Hz, 1H) 7.29-7.38 (m, 5H) 7.67-7.79 (m, 2H) 8.07 (d, J=5.5 Hz, 1H) 8.42 (d, J=8.1 Hz, 1H) 9.48 (s, 1H).

Table A below defines chemical designations for the substituents R₁, R₂ and R₃ for the compounds of formula I:

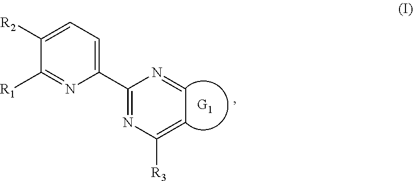

TABLE A
chemical designations for substituents
R₁, R₂ and R₃ of the compound of formula I:

| Line | R₁ | R₂ | R₃ |
|---|---|---|---|
| A.1.1 | phenyl | H | H |
| A.1.2 | phenyl | H | OH |
| A.1.3 | phenyl | H | Cl |
| A.1.4 | phenyl | H | CH₃ |
| A.1.5 | phenyl | H | OCH₃ |
| A.1.6 | phenyl | CH₃ | H |
| A.1.7 | phenyl | CH₃ | OH |
| A.1.8 | phenyl | CH₃ | Cl |
| A.1.9 | phenyl | CH₃ | CH₃ |
| A.1.10 | phenyl | CH₃ | OCH₃ |
| A.1.11 | phenoxy | H | H |
| A.1.12 | phenoxy | H | OH |
| A.1.13 | phenoxy | H | Cl |
| A.1.14 | phenoxy | H | CH₃ |
| A.1.15 | phenoxy | H | OCH₃ |
| A.1.16 | phenoxy | CH₃ | H |
| A.1.17 | phenoxy | CH₃ | OH |
| A.1.18 | phenoxy | CH₃ | Cl |
| A.1.19 | phenoxy | CH₃ | CH₃ |
| A.1.20 | phenoxy | CH₃ | OCH₃ |
| A.1.21 | phenylthio | H | H |
| A.1.22 | phenylthio | H | OH |
| A.1.23 | phenylthio | H | Cl |
| A.1.24 | phenylthio | H | CH₃ |
| A.1.25 | phenylthio | H | OCH₃ |
| A.1.26 | phenylthio | CH₃ | H |
| A.1.27 | phenylthio | CH₃ | OH |
| A.1.28 | phenylthio | CH₃ | Cl |
| A.1.29 | phenylthio | CH₃ | CH₃ |
| A.1.30 | phenylthio | CH₃ | OCH₃ |
| A.1.31 | benzyl | H | H |
| A.1.32 | benzyl | H | OH |
| A.1.33 | benzyl | H | Cl |
| A.1.34 | benzyl | H | CH₃ |
| A.1.35 | benzyl | H | OCH₃ |
| A.1.36 | benzyl | CH₃ | H |
| A.1.37 | benzyl | CH₃ | OH |
| A.1.38 | benzyl | CH₃ | Cl |
| A.1.39 | benzyl | CH₃ | CH₃ |
| A.1.40 | benzyl | CH₃ | OCH₃ |
| A.1.41 | C₆H₅CH═CH | H | H |
| A.1.42 | C₆H₅CH═CH | H | OH |
| A.1.43 | C₆H₅CH═CH | H | Cl |
| A.1.44 | C₆H₅CH═CH | H | CH₃ |
| A.1.45 | C₆H₅CH═CH | H | OCH₃ |
| A.1.46 | C₆H₅CH═CH | CH₃ | H |
| A.1.47 | C₆H₅CH═CH | CH₃ | OH |
| A.1.48 | C₆H₅CH═CH | CH₃ | Cl |
| A.1.49 | C₆H₅CH═CH | CH₃ | CH₃ |
| A.1.50 | C₆H₅CH═CH | CH₃ | OCH₃ |
| A.1.51 | C₆H₅C≡C | H | H |
| A.1.52 | C₆H₅C≡C | H | OH |
| A.1.53 | C₆H₅C≡C | H | Cl |
| A.1.54 | C₆H₅C≡C | H | CH₃ |
| A.1.55 | C₆H₅C≡C | H | OCH₃ |
| A.1.56 | C₆H₅C≡C | CH₃ | H |
| A.1.57 | C₆H₅C≡C | CH₃ | OH |
| A.1.58 | C₆H₅C≡C | CH₃ | Cl |
| A.1.59 | C₆H₅C≡C | CH₃ | CH₃ |
| A.1.60 | C₆H₅C≡C | CH₃ | OCH₃ |
| A.1.61 | 3-fluoro-4-methoxyphenyl | H | H |
| A.1.62 | 3-fluoro-4-methoxyphenyl | H | OH |
| A.1.63 | 3-fluoro-4-methoxyphenyl | H | Cl |
| A.1.64 | 3-fluoro-4-methoxyphenyl | H | CH₃ |
| A.1.65 | 3-fluoro-4-methoxyphenyl | H | OCH₃ |
| A.1.66 | 3-fluoro-4-methoxyphenyl | CH₃ | H |
| A.1.67 | 3-fluoro-4-methoxyphenyl | CH₃ | OH |
| A.1.68 | 3-fluoro-4-methoxyphenyl | CH₃ | Cl |
| A.1.69 | 3-fluoro-4-methoxyphenyl | CH₃ | CH₃ |
| A.1.70 | 3-fluoro-4-methoxyphenyl | CH₃ | OCH₃ |
| A.1.71 | 2-chlorobenzyl | H | H |
| A.1.72 | 2-chlorobenzyl | H | OH |
| A.1.73 | 2-chlorobenzyl | H | Cl |
| A.1.74 | 2-chlorobenzyl | H | CH₃ |
| A.1.75 | 2-chlorobenzyl | H | OCH₃ |
| A.1.76 | 2-chlorobenzyl | CH₃ | H |
| A.1.77 | 2-chlorobenzyl | CH₃ | OH |
| A.1.78 | 2-chlorobenzyl | CH₃ | Cl |
| A.1.79 | 2-chlorobenzyl | CH₃ | CH₃ |
| A.1.80 | 2-chlorobenzyl | CH₃ | OCH₃ |
| A.1.81 | 2-methylbenzyl | H | H |
| A.1.82 | 2-methylbenzyl | H | OH |
| A.1.83 | 2-methylbenzyl | H | Cl |
| A.1.84 | 2-methylbenzyl | H | CH₃ |
| A.1.85 | 2-methylbenzyl | H | OCH₃ |
| A.1.86 | 2-methylbenzyl | CH₃ | H |
| A.1.87 | 2-methylbenzyl | CH₃ | OH |
| A.1.88 | 2-methylbenzyl | CH₃ | Cl |
| A.1.89 | 2-methylbenzyl | CH₃ | CH₃ |
| A.1.90 | 2-methylbenzyl | CH₃ | OCH₃ |
| A.1.91 | 2,5-dimethylphenyl | H | H |
| A.1.92 | 2,5-dimethylphenyl | H | OH |
| A.1.93 | 2,5-dimethylphenyl | H | Cl |
| A.1.94 | 2,5-dimethylphenyl | H | CH₃ |
| A.1.95 | 2,5-dimethylphenyl | H | OCH₃ |
| A.1.96 | 2,5-dimethylphenyl | CH₃ | H |
| A.1.97 | 2,5-dimethylphenyl | CH₃ | OH |
| A.1.98 | 2,5-dimethylphenyl | CH₃ | Cl |
| A.1.99 | 2,5-dimethylphenyl | CH₃ | CH₃ |
| A.1.100 | 2,5-dimethylphenyl | CH₃ | OCH₃ |

Table 1: This table discloses the 100 compounds T1.1.1 to T1.1.100 of formula

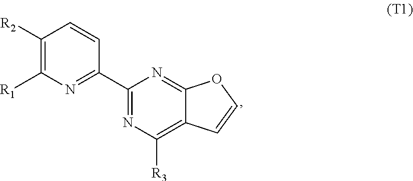

in which, for each of these 100 specific compounds, each of the of the variables R₁, R₂ and R₃ has the specific meaning given in the corresponding line, appropriately selected from the 100 lines A.1.1 to A.1. 100 of Table A. For example, the specific compound T1.1.23 is the compound of the formula T1, in which each of the of the variables R₁, R₂ and R₃ has the specific meaning given in the line A.1.23 of the Table A. According to the same system, also all of the other 100 specific compounds disclosed in the Table 1 as well as all of the specific compounds disclosed in the Tables 2 to 13 are specified analogously.

Table 2: This table discloses the 100 compounds T2.1.1 to T2.1.100 of the formula

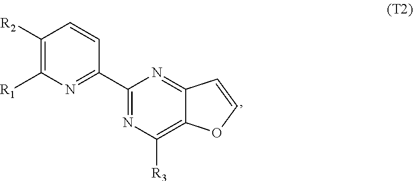

in which, for each of these 100 specific compounds, each of the of the variables R₁, R₂ and R₃ has the specific meaning given in the corresponding line, appropriately selected from the 100 lines A.1.1 to A.1.100 of the Table A.

Table 3: This table discloses the 100 compounds T3.1.1 to T3.1.100 of the formula

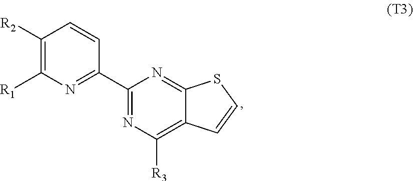

in which, for each of these 100 specific compounds, each of the of the variables R₁, R₂ and R₃ has the specific meaning given in the corresponding line, appropriately selected from the 100 lines A.1.1 to A.1.100 of the Table A.

Table 4: This table discloses the 100 compounds T4.1.1 to T4.1.100 of the formula

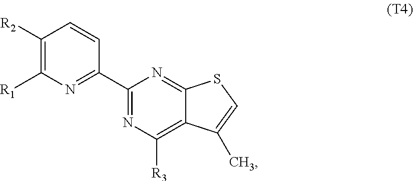

in which, for each of these 100 specific compounds, each of the of the variables R₁, R₂ and R₃ has the specific meaning given in the corresponding line, appropriately selected from the 100 lines A.1.1 to A.1.100 of the Table A.

Table 5: This table discloses the 100 compounds T5.1.1 to T5.1.100 of the formula

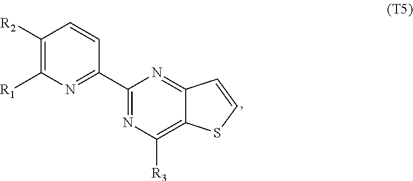

in which, for each of these 100 specific compounds, each of the of the variables R₁, R₂ and R₃ has the specific meaning given in the corresponding line, appropriately selected from the 100 lines A.1.1 to A.1.100 of the Table A.

Table 6: This table discloses the 100 compounds T6.1.1 to T6.1.100 of the formula

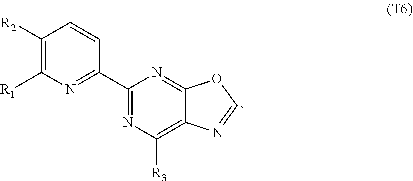

in which, for each of these 100 specific compounds, each of the of the variables R₁, R₂ and R₃ has the specific meaning given in the corresponding line, appropriately selected from the 100 lines A.1.1 to A.1.100 of the Table A.

Table 7: This table discloses the 100 compounds T7.1.1 to T7.1.100 of the formula

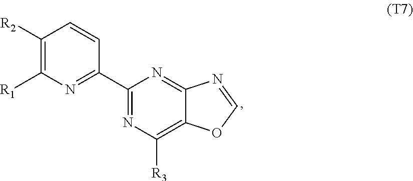

in which, for each of these 100 specific compounds, each of the of the variables R₁, R₂ and R₃ has the specific meaning given in the corresponding line, appropriately selected from the 100 lines A.1.1 to A.1.100 of the Table A.

Table 8: This table discloses the 100 compounds T8.1.1 to T8.1.100 of the formula

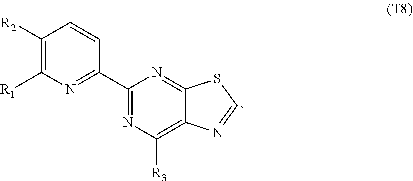

in which, for each of these 100 specific compounds, each of the of the variables R₁, R₂ and R₃ has the specific meaning given in the corresponding line, appropriately selected from the 100 lines A.1.1 to A.1.100 of the Table A.

Table 9: This table discloses the 100 compounds T9.1.1 to T9.1.100 of the formula

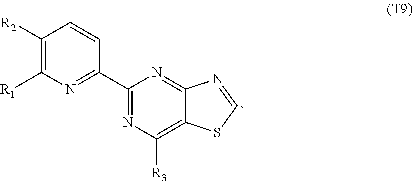

in which, for each of these 100 specific compounds, each of the of the variables R₁, R₂ and R₃ has the specific meaning given in the corresponding line, appropriately selected from the 100 lines A.1.1 to A.1.100 of the Table A.

Table 10: This table discloses the 100 compounds T10.1.1 to T10.1.100 of the formula

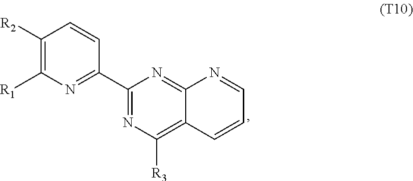

in which, for each of these 100 specific compounds, each of the of the variables R₁, R₂ and R₃ has the specific meaning given in the corresponding line, appropriately selected from the 100 lines A.1.1 to A.1.100 of the Table A.

Table 11: This table discloses the 100 compounds T11.1.1 to T11.1.100 of the formula

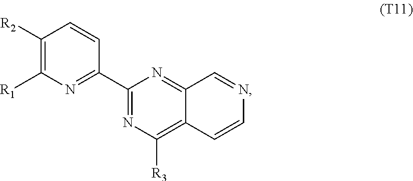

in which, for each of these 100 specific compounds, each of the of the variables R₁, R₂ and R₃ has the specific meaning given in the corresponding line, appropriately selected from the 100 lines A.1.1 to A.1.100 of the Table A.

Table 12: This table discloses the 100 compounds T12.1.1 to T12.1.100 of the formula

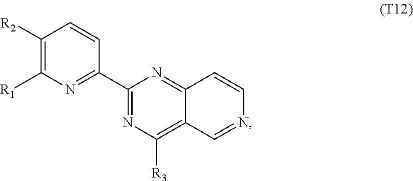

in which, for each of these 100 specific compounds, each of the of the variables R₁, R₂ and R₃ has the specific meaning given in the corresponding line, appropriately selected from the 100 lines A.1.1 to A.1.100 of the Table A.

Table 13: This table discloses the 100 compounds T13.1.1 to T13.1.100 of the formula

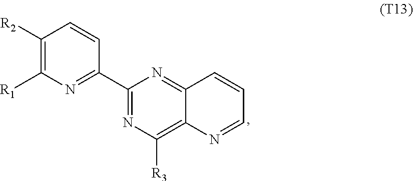

in which, for each of these 100 specific compounds, each of the of the variables R₁, R₂ and R₃ has the specific meaning given in the corresponding line, appropriately selected from the 100 lines A.1.1 to A.1.100 of the Table A.

Table 14 shows selected m.p. data and selected LC/MS data for compounds of Table 1 to Table 13.

Throughout this description, temperatures are given in degrees Celsius and “m.p.” means melting point. LC/MS means Liquid Chromatography Mass Spectroscopy and the description of the apparatus and the method is: (HP 1100 HPLC from Agilent, Phenomenex Gemini C18, 3 μm (micro meter) particle size, 110 Angström, 30×3 mm column, 1.7 mL/min., 60° C., H₂O+0.05% HCOOH (95%)/CH₃CN/MeOH 4:1+0.04% HCOOH (5%)—2 min.—CH₃CN/MeOH 4:1+0.04% HCOOH (5%)—0.8 min., ZQ Mass Spectrometer from Waters, ionization method: electrospray (ESI), Polarity: positive ions, Capillary (kV) 3.00, Cone (V) 30.00, Extractor (V) 2.00, Source Temperature (° C.) 100, Desolvation Temperature (° C.) 250, Cone Gas Flow (L/Hr) 50, Desolvation Gas Flow (L/Hr) 400)).

TABLE 14
Melting point and LC/MS data for compounds of Table 1 to 13:

| Compound | Melting point | |
|---|---|---|
| No. | (° C.) | LC/MS |
| T3.1.31 | | Rt = 1.72 min.; MS: m/z = 304 (M + 1) |
| T3.1.32 | | Rt = 1.91 min.; MS: m/z = 320 (M + 1) |
| T3.1.33 | | Rt = 1.98 min.; MS: m/z = 338 (M + 1) |
| T3.1.34 | | Rt = 1.79 min.; MS: m/z = 318 (M + 1) |
| T3.1.35 | | Rt = 1.80 min.; MS: m/z = 334 (M + 1) |
| T3.1.54 | 105-111 | Rt = 2.03 min.; MS: m/z = 327 (M + 1) |
| T3.1.66 | | Rt = 1.82 min.; MS: m/z = 352 (M + 1) |
| T4.1.31 | | Rt = 1.81 min.; MS: m/z = 318 (M + 1) |
| T10.1.6 | 199-201 | |
| T13.1.6 | 217-218 | |
| T13.1.9 | 159-161 | |
| T13.1.31 | 118-120 | |

Table 15 shows selected m.p. data and selected LC/MS data for compounds of structure (I) where G₁, R₁, R₂, R₃ and R₄ are as defined for formula (I).

TABLE 15
Melting point and LC/MS data for compounds of formula (I):

| | | Melting | |
|---|---|---|---|
| Entry | Compound | point (° C.) | LC/MS |
| T15.1 | 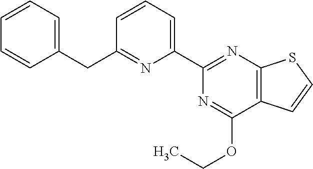 | | Rt = 1.98 min.; MS: m/z = 348 (M + 1) |
| T15.2 | 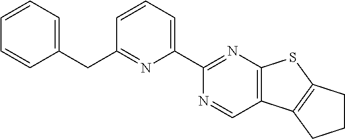 | 120-128 | |
| T15.3 | 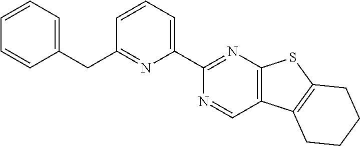 | 133-137 | |
| T15.4 | 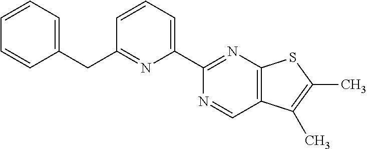 | 97-99 | |
| T15.5 | 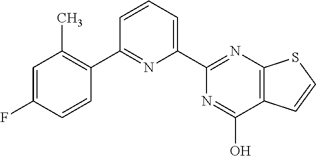 | 166-171 | |
| T15.6 | 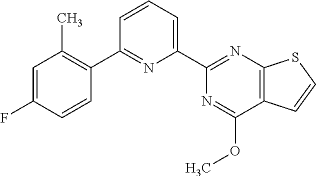 | 198-202 | |
| T15.7 | 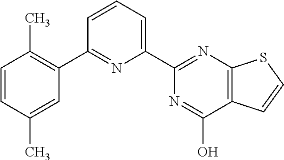 | 164-170 | |
| T15.8 | 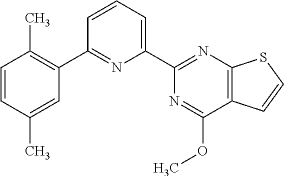 | 110-114 | |
| T15.9 | 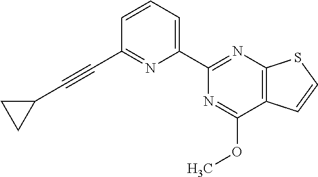 | | Rt = 1.91 min.; MS: m/z = 308 (M + 1) |
| T15.10 | 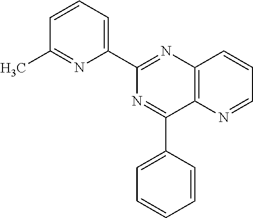 | 129-132 | |
| T15.11 | 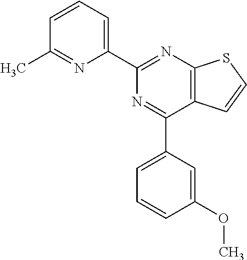 | 158-161 | |
| T15.12 | 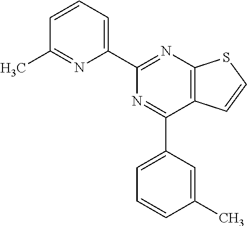 | 98-102 | |
| T15.13 | 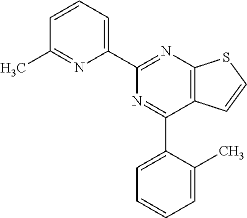 | 187-191 | |
| T15.14 | 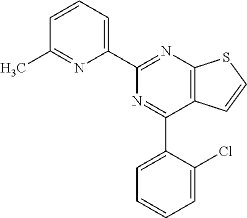 | 181-184 | |
| T15.15 | 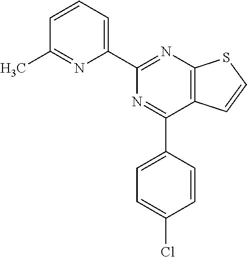 | 180-181 | |
| T15.16 | 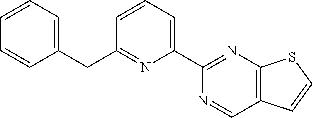 | 109-112 | |
| T15.17 | 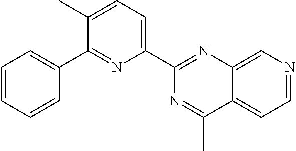 | 185-186 | Rt = 1.66 min.; MS: m/z = 333 (M + 1) |
| T15.18 | 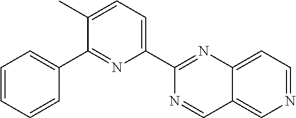 | | Rt = 1.81 min.; MS: m/z = 333 (M + 1) |

Formulation examples for compounds of formula I:

### Example F-1.1 to F-1.2

### Emulsifiable Concentrates

| Components | F-1.1 | F-1.2 |
|---|---|---|
| compound of Tables 1-13 | 25% | 50% |
| calcium dodecylbenzenesulfonate | 5% | 6% |
| castor oil polyethylene glycol ether | 5% | — |
| (36 mol ethylenoxy units) | | |
| tributylphenolpolyethylene glycol ether | — | 4% |
| (30 mol ethylenoxy units) | | |
| cyclohexanone | — | 20% |
| xylene mixture | 65% | 20% |

Emulsions of any desired concentration can be prepared by diluting such concentrates with water.

### Example F-2

### Emulsifiable Concentrate

| Components | F-2 |
|---|---|
| compound of Tables 1-13 | 10% |
| octylphenolpolyethylene glycol ether | 3% |
| (4 to 5 mol ethylenoxy units) | |
| calcium dodecylbenzenesulfonate | 3% |
| castor oil polyglycol ether | 4% |
| (36 mol ethylenoxy units) | |
| cyclohexanone | 30% |
| xylene mixture | 50% |

Emulsions of any desired concentration can be prepared by diluting such concentrates with water.

### Examples F-3.1 to F-3.4

### Solutions

| Components | F-3.1 | F-3.2 | F-3.3 | F-3.4 |
|---|---|---|---|---|
| compound of Tables 1-13 | 80% | 10% | 5% | 95% |
| Propylene glycol monomethyl ether | 20% | — | | — |
| polyethylene glycol | — | 70% | | — |
| (relative molecular mass: | | | | |
| 400 atomic mass units) | | | | |
| N-methylpyrrolid-2-one | — | 20% | — | — |
| epoxidised coconut oil | — | — | 1% | 5% |
| benzin (boiling range: 160-190°) | — | — | — | 94% |

The solutions are suitable for use in the form of microdrops.

### Examples F-4.1 to F-4.4

### Granulates

| Components | F-4.1 | F-4.2 | F-4.3 | F-4.4 |
|---|---|---|---|---|
| compound of Tables 1-13 | 5% | 10% | 8% | 21% |
| kaolin | 94% | — | 79% | 54% |
| highly dispersed silicic acid | 1% | — | 13% | 7% |
| attapulgite | — | 90% | — | 18% |

The novel compound is dissolved in dichloromethane, the solution is sprayed onto the carrier and the solvent is then removed by distillation under vacuum.

### Examples F-5.1 and F-5.2

### Dusts

| Components | F-5.1 | F-5.2 |
|---|---|---|
| compound of Tables 1-13 | 2% | 5% |
| highly dispersed silicic acid | 1% | 5% |
| talcum | 97% | — |
| kaolin | — | 90% |

Ready for use dusts are obtained by intimately mixing all components.

### Examples F-6.1 to F-6.3

### Wettable Powders

| Components | F-6.1 | F-6.2 | F-6.3 |
|---|---|---|---|
| compound of Tables 1-13 | 25% | 50% | 75% |
| sodium lignin sulfonate | 5% | 5% | — |
| sodium lauryl sulfate | 3% | — | 5% |
| sodium diisobutylnaphthalene sulfonate | — | 6% | 10% |
| octylphenolpolyethylene glycol ether | — | 2% | — |
| (7 to 8 mol ethylenoxy units) | | | |
| highly dispersed silicic acid | 5% | 10% | 10% |
| kaolin | 62% | 27% | — |

All components are mixed and the mixture is thoroughly ground in a suitable mill to give wettable powders which can be diluted with water to suspensions of any desired concentration.

### Example F7

### Flowable Concentrate for Seed Treatment

| Components | F-7.1 |
|---|---|
| compound of Tables 1-13 | 40% |
| Propylene glycol | 5% |
| copolymer butanol PO/EO | 2% |
| tristyrenephenole with 10-20 moles EO | 2% |
| 1,2-benzisothiazolin-3-one | 0.5% |
| (in the form of a 20% solution in water) | |
| monoazo-pigment calcium salt | 5% |
| Silicone oil | 0.2% |
| (in the form of a 75% emulsion in water) | |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Biological Examples

### Example B1

### Fungicidal Activity Against Alternaria solani/Tomato/Leaf Disc (Early Blight)

Tomato leaf disks cv. Baby were placed on agar in multiwell plates (24-well format) and sprayed with the formulated test compound diluted in water. The leaf disks were inoculated with a spore suspension of the fungus 2 days after application. The inoculated leaf disks were incubated at 23° C./21° C. (day/night) and 80% rh under a light regime of 12/12 h (light/dark) in a climate cabinet and the activity of a compound was assessed as percent disease control compared to untreated when an appropriate level of disease damage appears on untreated check disk leaf disks (5-7 days after application). Compounds T3.1.9, T3.1.66 and T10.1.6 at 200 ppm give at least 80% disease control in this test when compared to untreated control leaf disks under the same conditions, which show extensive disease development.

### Example B2

### Fungicidal Activity Against Blumeria graminis F. Sp. Tritici (Erysiphe graminis F. Sp. tritici)/Wheat/Leaf Disc Preventative (Powdery Mildew on Wheat)

Wheat leaf segments cv. Kanzler were placed on agar in a multiwell plate (24-well format) and sprayed with the formulated test compound diluted in water. The leaf disks were inoculated by shaking powdery mildew infected plants above the test plates 1 day after application. The inoculated leaf disks were incubated at 20° C. and 60% rh under a light regime of 24 h darkness followed by 12 h light/12 h darkness in a climate chamber and the activity of a compound was assessed as percent disease control compared to untreated when an appropriate level of disease damage appears on untreated check leaf segments (6-8 days after application).

Compounds T3.1.9, T3.1.31, T3.1.34, T3.1.66, T15.15 and T15.16 at 200 ppm give at least 80% disease control in this test when compared to untreated control leaf disks under the same conditions, which show extensive disease development.

### Example B3

### Fungicidal Activity Against Botryotinia fuckeliana (Botrytis cinerea)/Liquid Culture (Gray Mould)

Conidia of the fungus from cryogenic storage were directly mixed into nutrient broth (Vogels broth). After placing a (DMSO) solution of test compound into a microtiter plate (96-well format), the nutrient broth containing the fungal spores was added. The test plates were incubated at 24° C. and the inhibition of growth was determined photometrically 3-4 days after application.

Compounds T3.1.9, T3.1.31, T3.1.34, T3.1.66, T4.1.31, T10.1.6, T13.1.6, T13.1.31, T15.4, T15.5, T15.10, T15.11, T15.12, T15.13, T15.14, T15.15 and T15.16 at 200 ppm give at least 80% disease control in this test when compared to untreated control leaf disks under the same conditions, which show extensive disease development.

### Example B4

### Fungicidal Activity Against Gaeumannomyces graminis/Liquid Culture (Take-all of Cereals)

Mycelial fragments of the fungus from cryogenic storage were directly mixed into nutrient broth (PDB potato dextrose broth). After placing a (DMSO) solution of test compound into a microtiter plate (96-well format), the nutrient broth containing the fungal spores is added. The test plates were incubated at 24° C. and the inhibition of growth was determined photometrically 4-5 days after application.

Compounds T3.1.9, T3.1.31, T3.1.33, T3.1.34, T3.1.35, T3.1.54, T3.1.66, T4.1.31, T10.1.6, T13.1.6, T13.1.31, T15.1, T15.2, T15.4, T15.5, T15.7, T15.8, T15.9, T15.10, T15.11, T15.12, T15.13, T15.14 T15.15 and T15.16 at 200 ppm give at least 80% disease control in this test when compared to untreated control leaf disks under the same conditions, which show extensive disease development.

### Example B5

### Fungicidal Activity Against Glomerella lagenarium (Colletotrichum lagenarium)/Liquid Culture (Anthracnose)

Conidia of the fungus from cryogenic storage were directly mixed into nutrient broth (PDB potato dextrose broth). After placing a (DMSO) solution of test compound into a microtiter plate (96-well format), the nutrient broth containing the fungal spores was added. The test plates were incubated at 24° C. and the inhibition of growth was measured photometrically 3-4 days after application.

Compounds T3.1.9, T3.1.31, T3.1.33, T3.1.34, T3.1.35, T3.1.54, T3.1.66, T4.1.31, T10.1.6, T13.1.31, T15.1, T15.10, T15.11, T15.12, T15.13, T15.14, T15.15 and T15.16 at 200 ppm give at least 80% disease control in this test when compared to untreated control leaf disks under the same conditions, which show extensive disease development.

### Example B6

### Fungicidal Activity Against Monographella nivalis (Microdochium nivale)/Liquid Culture (Foot Rot Cereals)

Conidia of the fungus from cryogenic storage were directly mixed into nutrient broth (PDB potato dextrose broth). After placing a (DMSO) solution of test compound into a microtiter plate (96-well format), the nutrient broth containing the fungal spores was added. The test plates were incubated at 24° C. and the inhibition of growth was determined photometrically 4-5 days after application.

Compounds T3.1.31, T3.1.33, T3.1.34, T3.1.35, T3.1.54, T4.1.31, T10.1.6, T13.1.6, T13.1.31, T15.1, T15.4, T15.7, T15.9, T15.11, T15.12, T15.15 and T15.16 at 200 ppm give at least 80% disease control in this test when compared to untreated control leaf disks under the same conditions, which show extensive disease development.

### Example B7

### Fungicidal Activity Against Mycosphaerella arachidis (Cercospora arachidicola)/Liquid Culture (Early Leaf Spot)

Conidia of the fungus from cryogenic storage were directly mixed into nutrient broth (PDB potato dextrose broth). After placing a (DMSO) solution of test compound into a microtiter plate (96-well format), the nutrient broth containing the fungal spores was added. The test plates were incubated at 24° C. and the inhibition of growth was determined photometrically 4-5 days after application.

Compounds T3.1.9, T3.1.31, T3.1.33, T3.1.34, T3.1.35, T3.1.54, T3.1.66, T4.1.31, T10.1.6, T13.1.6, T13.1.31, T15.1, T15.2, T15.4, T15.8, T15.9, T15.10, T15.11, T15.12, T15.13, T15.14, T15.15 and T15.16 at 200 ppm give at least 80% disease control in this test when compared to untreated control leaf disks under the same conditions, which show extensive disease development.

### Example B8

### Fungicidal Activity Against Mycosphaerella graminicola (Septoria tritici)/Liquid Culture (Septoria Blotch)

Conidia of the fungus from cryogenic storage were directly mixed into nutrient broth (PDB potato dextrose broth). After placing a (DMSO) solution of test compound into a microtiter plate (96-well format), the nutrient broth containing the fungal spores was added. The test plates were incubated at 24° C. and the inhibition of growth was determined photometrically 4-5 days after application.

Compounds T3.1.9, T3.1.31, T3.1.33, T3.1.34, T3.1.35, T3.1.54, T3.1.66, T4.1.31, T10.1.6, T13.1.31, T15.1, T15.8, T15.10, T15.11, T15.12, T15.13, T15.14, T15.15 and T15.16 at 200 ppm give at least 80% disease control in this test when compared to untreated control leaf disks under the same conditions, which show extensive disease development.

### Example B9

### Fungicidal Activity Against Phaeosphaeria nodorum (Septoria nodorum)/Wheat/Leaf Disc Preventative (Glume Blotch)

Wheat leaf segments cv. Kanzler were placed on agar in a multiwell plate (24-well format) and sprayed with the formulated test compound diluted in water. The leaf disks were inoculated with a spore suspension of the fungus 2 days after application. The inoculated test leaf disks were incubated at 20° C. and 75% rh under a light regime of 12 h light/12 h darkness in a climate cabinet and the activity of a compound was assessed as percent disease control compared to untreated when an appropriate level of disease damage appears in untreated check leaf disks (5-7 days after application).

Compounds T3.1.9, T3.1.31, T3.1.33, T3.1.34, T3.1.35, T3.1.54, T3.1.66, T4.1.31, T10.1.6, T13.1.31, T15.10, T15.11, T15.12, T15.13, T15.14, T15.15 and T15.16 at 200 ppm give at least 80% disease control in this test when compared to untreated control leaf disks under the same conditions, which show extensive disease development.

### Example B10

### Fungicidal Activity Against Phytophthora infestans/Tomato/Leaf Disc Preventative (Late Blight)

Tomato leaf disks were placed on water agar in multiwell plates (24-well format) and sprayed with the formulated test compound diluted in water. The leaf disks were inoculated with a spore suspension of the fungus 1 day after application. The inoculated leaf disks were incubated at 16° C. and 75% rh under a light regime of 24 h darkness followed by 12 h light/12 h darkness in a climate cabinet and the activity of a compound was assessed as percent disease control compared to untreated when an appropriate level of disease damage appears in untreated check leaf disks (5-7 days after application).

Compounds T3.1.31 at 200 ppm give at least 80% disease control in this test when compared to untreated control leaf disks under the same conditions, which show extensive disease development.

### Example B11

### Fungicidal Activity Against Plasmopara viticola/Grape/Leaf Disc Preventative (Late Blight)

Grape vine leaf disks were placed on water agar in multiwell plates (24-well format) and sprayed with the formulated test compound diluted in water. The leaf disks were inoculated with a spore suspension of the fungus 1 day after application. The inoculated leaf disks were incubated at 19° C. and 80% rh under a light regime of 12 h light/12 h darkness in a climate cabinet and the activity of a compound was assessed as percent disease control compared to untreated when an appropriate level of disease damage appears in untreated check leaf disks (6-8 days after application).

Compounds T3.1.31 and T3.1.33 at 200 ppm give at least 80% disease control in this test when compared to untreated control leaf disks under the same conditions, which show extensive disease development.

### Example B12

### Fungicidal Activity Against Puccinia recondita f. sp. tritici/Wheat/Leaf Disc Preventative (Brown Rust)

Wheat leaf segments cv. Kanzler were placed on agar in multiwell plates (24-well format) and sprayed with the formulated test compound diluted in water. The leaf disks were inoculated with a spore suspension of the fungus 1 day after application. The inoculated leaf segments were incubated at 19° C. and 75% rh under a light regime of 12 h light/12 h darkness in a climate cabinet and the activity of a compound was assessed as percent disease control compared to untreated when an appropriate level of disease damage appears in untreated check leaf segments (7-9 days after application).

Compounds T3.1.9, T3.1.31, T3.1.33, T3.1.34, T4.1.31, T10.1.6, T15.1, T15.10, T15.11, T15.12, T15.15 and T15.16 at 200 ppm give at least 80% disease control in this test when compared to untreated control leaf disks under the same conditions, which show extensive disease development.

### Example B13

### Fungicidal Activity Against Pyrenophora teres/Barley/Leaf Disc Preventative (Net Blotch)

Barley leaf segments cv. Hasso were placed on agar in a multiwell plate (24-well format) and sprayed with the formulated test compound diluted in water. The leaf segmens were inoculated with a spore suspension of the fungus 2 days after application. The inoculated leaf segments were incubated at 20° C. and 65% rh under a light regime of 12 h light/12 h darkness in a climate cabinet and the activity of a compound was assessed as disease control compared to untreated when an appropriate level of disease damage appears in untreated check leaf segments (5-7 days after application).

Compounds T3.1.9, T3.1.31, T3.1.34, T3.1.66, T4.1.31, T10.1.6, T13.1.31, T15.4, T15.8, T15.10, T15.11, T15.12, T15.14, T15.15 and T15.16 at 200 ppm give at least 80% disease control in this test when compared to untreated control leaf disks under the same conditions, which show extensive disease development.

### Example B14

### Fungicidal Activity Against Thanatephorus cucumeris (Rhizoctonia solani)/Liquid Culture (Foot Rot, Damping-Off)

Mycelia fragments of a newly grown liquid culture of the fungus were directly mixed into nutrient broth (PDB potato dextrose broth). After placing a (DMSO) solution of the test compounds into a microtiter plate (96-well format), the nutrient broth containing the fungal material was added. The test plates were incubated at 24° C. and the inhibition of growth was determined photometrically 3-4 days after application.

Compounds .1.9, T3.1.31, T3.1.34, T3.1.54, T3.1.66, T4.1.31, T10.1.6, T15.11, T15.12, T15.15 and T15.16 at 200 ppm give at least 80% disease control in this test when compared to untreated control leaf disks under the same conditions, which show extensive disease development.

1. A compound of formula I 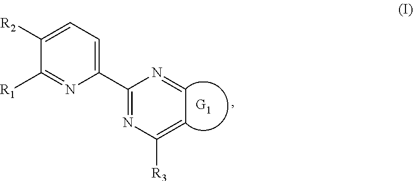
wherein
G₁ represents together with the two ring atoms of the pyrimidine ring to which it is attached, a 5- to 6-membered aromatic heterocyclic ring system which contains one or two heteroatoms selected from the group consisting of nitrogen, oxygen and sulfur; and wherein said 5- to 6-membered aromatic heterocyclic ring system can be mono- or disubstituted by substituents selected from the group consisting of C₁-C₆alkyl, C₁-C₆alkoxy, C₁-C₆alkylthio and halogen or two adjacent substituents together represent a polymethylene group of the formula —(CH₂)ₘ—in which m is 3 or 4;
R₁ is C₁-C₆alkyl, C₁-C₆haloalkyl, or a group —X—R₄, wherein X is a bond, oxygen, sulfur, C₁-C₄alkylene, C₂-C₄alkenylene or C₂-C₄alkynylene;
R₂ is hydrogen, C₁-C₆alkyl, C₁-C₆haloalkyl, C₁-C₆alkoxy, C₁-C₆haloalkoxy or C₃-C₈cycloalkyl;
R₃ is hydrogen, hydroxy, halogen, C₁-C₆alkyl, C₁-C₆alkoxy, phenyl, benzyl; or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;
R₄ is phenyl which can be mono-, di- or trisubstituted by substituents selected from the group consisting of halogen, nitro, C₁-C₄alkyl, C₁-C₆haloalkyl, C₁-C₄alkoxy and C₁-C₆haloalkoxy;
or
R₄ is additionally C₂-C₆alkynyl if X is a bond;
and agronomically acceptable salts or isomers or structural isomers or stereoisomers ordiastereoisomers or enantiomers or tautomers or atropoisomers and N-oxides of those compounds,
with the proviso that the compounds 4-chloro-5-methyl-2-(6-methyl-2-pyridinyl)-Thieno[2,3-d]pyrimidine and 4-chloro-2-(6-methyl-2-pyridinyl)-Thieno[3,2-d]pyrimidine and 5-methyl-2-(6-methyl-2-pyridinyl)-Thieno[2,3-d]pyrimidin-4(1H)-one and 2-(6-methyl-2-pyridinyl)-Thieno[3,2-d]pyrimidin-4(1H)-one and 4-chloro-2-(6-methyl-2-pyridinyl)-7H-Pyrrolo[2,3-d]pyrimidine and 4-iodo-2-(6-methyl-2-pyridinyl)-7H-Pyrrolo[2,3-d]pyrimidine and 2-(6-methyl-2-pyridinyl)-4(3H)-Pteridinone and 4-chloro-2-(6-methyl-2-pyridinyl)-Thieno[3,2-d]pyrimidine and 4-chloro-5-methyl-2-(6-methyl-2-pyridinyl)-Thieno[2,3-d]pyrimidine and 2-(6-methyl-2-pyridinyl)-Thieno[3,2-d]pyrimidin-4(3H)-one and 5-methyl-2-(6-methyl-2-pyridinyl)-Thieno[2,3-d]pyrimidin-4(1H)-one are excluded from the scope of protection.

2. A compound of formula I according to claim 1, wherein the pyrimidine ring together with the substituent G₁ forms a ring system selected from the group consisting of 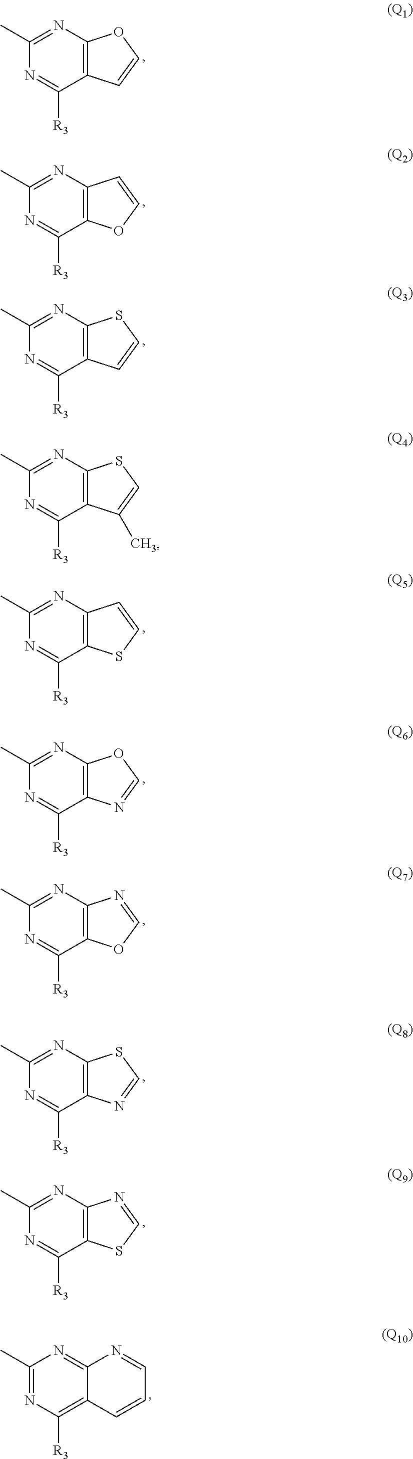 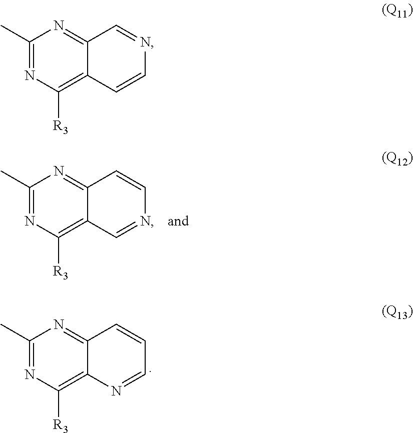
wherein R₃ has the meaning as defined under formula I in claim 1.

3. A compound of formula I according to claim 2, wherein R₃ is hydrogen, C₁-C₄alkyl, hydroxy, halogen or C₁-C₄alkoxy.

4. A compound of formula I according to claim 2, wherein the ring system is selected from Q₃, Q₄. Q₁₀, Q₁₁, Q₁₂ and Q₁₃.

5. A compound of formula I according to claim 1, wherein
R₁ is a group —X—R₄, wherein X is a bond or C₁-C₄alkylene; and R₄ is phenyl which can be mono- or di- or trisubstituted by substituents selected from the group consisting of halogen, nitro, C₁-C₄alkyl, C₁-C₆haloalkyl, C₁-C₄alkoxy and C₁-C₆haloalkoxy.

6. A compound of formula I according to claim 1, wherein
R₁ is a group —X—R₄, wherein X is a bond or C₁-C₄alkylene.

7. A compound of formula I according to claim 1, wherein R₄ is phenyl which can be mono- or di- or trisubstituted by substituents selected from the group consisting of halogen, nitro, C₁-C₄alkyl, C₁-C₆haloalkyl, C₁-C₄alkoxy and C₁-C₆haloalkoxy.

8. A compound of formula I according to claim 1, wherein
R₁ is phenyl which can be substituted by halogen or C₁-C₄alkoxy, or R₁ is benzyl;
R₂ is hydrogen or C₁-C₄alkyl; and
R₃ is hydrogen, C₁-C₄alkyl, hydroxy, halogen or C₁-C₄alkoxy.

9. A compound of formula I according to claim 1, wherein
R₁ is methyl or a group —X—R₄, wherein X is a bond, oxygen, sulfur, C₁-C₄alkylene, C₂-C₄alkenylene or C₂-C₄alkynylene; and R₄ is phenyl which can be mono-, di- or trisubstituted by substituents selected from the group consisting of halogen, nitro, C₁-C₄alkyl, C₁-C₆haloalkyl, C₁-C₄alkoxy and C₁-C₆haloalkoxy; or
R₄ is additionally C₂-C₆alkynyl if X is a bond;

10. A compound of formula I according to claim 9, wherein
R₁ is methyl;
R₂ is hydrogen or C₁-C₄alkyl;
R₃ is C₁-C₄alkyl, C₁-C₄alkoxy or phenyl or benzyl which is mono-, di- or trisubstituted by substituents selected from the group consisting of halogen, nitro, C₁-C₄alkyl, C₁-C₆haloalkyl, C₁-C₄alkoxy and C₁-C₆haloalkoxy;

11. A method of controlling or preventing infestation of useful plants by phytopathogenic microorganisms, wherein a compound of formula I according to claim 1 or a composition, comprising this compound as active ingredient, is applied to the plants, to parts thereof or the locus thereof.

12. A composition for controlling and protecting against phytopathogenic microorganisms, comprising a compound of formula I according to claim 1 and at least one auxiliary.
